(19) United States
(12) Patent Application Publication
(10) Pub. No.: US 2021/0115071 A1
(43) Pub. Date: Apr. 22, 2021

(54) NANOSCALE METAL-ORGANIC FRAMEWORKS FOR ENHANCED TUMOR CHEMORADIATION

(71) Applicant: Oregon State University, Corvallis, OR (US)

(72) Inventors: Conroy Ghin Chee Sun, Corvallis, OR (US); Megan Judith Neufeld, Corvallis, OR (US); Allison Nicole DuRoss, Corvallis, OR (US)

(21) Appl. No.: 17/072,895

(22) Filed: Oct. 16, 2020

Related U.S. Application Data
(60) Provisional application No. 62/916,590, filed on Oct. 17, 2019.
(60) Provisional application No. 62/931,026, filed on Nov. 5, 2019.

(51) Int. Cl. C07F 7/00; A61K 9/51; A61K 31/655; A61K 31/166; A61K 31/55; A61K 31/5025; A61K 31/454; A61K 31/501

(52) U.S. Cl. CPC C07F 7/00; A61K 9/5161; A61K 31/655; A61K 31/166; B82Y 5/00; A61K 31/5025; A61K 31/454; A61K 31/501; A61K 9/5115; A61K 31/55

(57) ABSTRACT

Compositions and methods for treatment of cancers are disclosed. The compositions and methods provide synergistic strategy for enhancing the therapeutic efficacy of radiotherapy (RT) via nanoscale metal-organic frameworks (nMOFs)-mediated drug delivery. Exemplary nMOF containing the high-Z element Hf and the ligand 1,4-dicarboxybenzene (Hf-BDC) were synthesized and loaded with DNA damage repair (DDR) inhibitor and DNA-targeting drugs. Synergistic enhancement was demonstrated in vivo where the combination of concurrent radiation with intravenous administration of exemplary nMOF resulted in improved tumor control and increased apoptosis with no apparent toxicity.

### CROSS-REFERENCES TO RELATED APPLICATIONS

This application claims the benefit of U.S. Provisional Application 62/916,590, filed Oct. 17, 2019, and U.S. Provisional Application 62/931,026, filed Nov. 5, 2019, the disclosures of which are incorporated herein by reference in their entirety.

### STATEMENT OF GOVERNMENT LICENSE RIGHTS

This invention was made with Government support under Contract No. 5R35GM119839-04 awarded by the National Institutes of Health. The government has certain rights in the invention.

### BACKGROUND

Radiation therapy (RT) is one of the most utilized and efficacious treatment modalities in clinical oncology. However, the full potential of RT is limited due to the narrow therapeutic window of X-ray radiation. In most cases, optimal treatment outcomes are constrained by a balance between the dose required for therapeutic efficacy and the amount of damage that can be sustained by the surrounding healthy tissue. Recent technological advances associated with radiation dose planning and instrumentation have improved delivery accuracy, but off target toxicity remains a challenge in RT. In addition to physics and computational advances, multi-modal therapeutic strategies such as concurrent administration of systemic chemotherapeutics with RT (chemoradiation) are often employed to improve patient outcomes. Despite its success, universal adoption of chemoradiation is hindered by nonspecific drug distribution, undesirable pharmacokinetics, and the resulting undesirable combined toxicities. Like traditional RT, chemoradiation treatment regimens are similarly limited by the balance between efficacy and toxicity. At the same time, advances in drug delivery have presented a unique solution to overcome these ubiquitous limitations by utilizing nanotechnology to deliver chemotherapeutics during a course of RT.

A need exists for therapeutic nanomaterials that can augment RT and maintain cellular damage at lower administered radiation doses.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a composition comprising a hybrid material comprising nanosized metal-organic-framework (nMOF) and two or more therapeutic agents selected from the group consisting of DNA-targeting chemotherapeutic agents, DNA Damage Repair (DDR) inhibitors, and a combination thereof. In some embodiments, the two or more therapeutic agents comprise a combination of one or more DNA-targeting chemotherapeutic agents and one or more DNA Damage Repair (DDR) inhibitors. In some embodiments, the one or more DNA-targeting chemotherapeutic agents is hydrophobic. In some embodiments, the one or more one or more DNA Damage Repair (DDR) inhibitors is hydrophobic.

In some embodiments, the one or more DNA-targeting chemotherapeutic agents is a platinum agent, a triazene, a nitrosourea, an alkylating hexitol, or a nitrogen mustard. In some embodiments, the one or more DNA-targeting chemotherapeutic agents is dacarbazine, procarbazine, or temozolomide. In some embodiments, the one or more one or more DNA Damage Repair (DDR) inhibitors is a tyrosine kinase inhibitor or a poly (ADP-ribose) polymerase (PARP) inhibitor. In some embodiments, the one or more one or more DNA Damage Repair (DDR) inhibitors is rucaparib, talazoparib, niraparib, or olaparib.

In some embodiments, the nanosized metal-organic-framework (nMOF) comprises Hf and 1,4-benzenedicarboxylic acid (Hf-BDC).

In some embodiments, the nanosized metal-organic-framework (nMOF) is PEGylated.

In some embodiments, the nanosized metal-organic-framework (nMOF) is coated with an agent that has affinity to a cell surface protein upregulated under inflammatory conditions. In some embodiments, the nanosized metal-organic-framework (nMOF) is coated with a polysaccharide. In some embodiments, the polysaccharide has affinity for P-Selectin. In some embodiments, the polysaccharide is fucoidan or P-selectin glycoprotein ligand 1.

In another aspect, the disclosure provides a method of treatment of cancer in a subject, comprising administering to a subject in need thereof an effective therapeutic dose of a composition disclosed herein.

In some embodiments, the composition is administered intravenously or intratumorally. In some embodiments, the method further comprises administering a therapeutically effective dose of radiation to the patient. In some embodiments, the administering a therapeutically effective dose of radiation to the patient is done prior to, concurrently with, or after the administration of the composition.

In some embodiments, the cancer is colorectal cancer, breast cancer, head and neck cancer, esophageal cancer, gastric cancer, lung cancer, pancreatic cancer, gastric cancer, bladder cancer, cervical cancer, or a brain tumor.

In another aspect, the disclosure provides a method of treatment of tumor in a subject, comprising administering a therapeutically effective dose of radiation to the tumor, wherein the therapeutically effective dose of radiation is a dose that results in increased expression of a cell surface protein upregulated under inflammatory conditions, and administering a therapeutically effective dose of a composition comprising a hybrid material comprising nanosized metal-organic-framework (nMOF) and two or more therapeutic agents selected from the group consisting of DNA-targeting chemotherapeutic agents, DNA Damage Repair (DDR) inhibitors, and a combination thereof, and wherein the nMOF is coated with an agent that has affinity to a cell surface protein upregulated under inflammatory conditions.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A and 1B are a schematic illustration of an exemplary composition TB@Hf-BDC-PEG as a multi-modal platform for chemoradiation therapy. FIG. 1A depicts the first mechanism of therapeutic activity includes radiosensitization through hafnium-mediated ROS enhancement following irradiation whereas the second mechanism involves the delivery of DNA damage repair (DDR) inhibitors. FIG. 1B shows that on the cellular level, TB@Hf-BDC-PEG administration and tumor cell internalization enhance RT induced cellular damage by preferentially absorbing X-rays and amplifying ROS generation. Following RT, the corresponding release of the DDR inhibitors from the nMOF pores aids in preventing the repair of the damaged DNA.

FIG. 2 depicts schematic synthesis route of an exemplary composition TB@Hf-BDC-PEG.

FIGS. 3A-3F show characterization of exemplary Hf-BDC nMOFs. FIG. 3A shows representative TEM images of Hf-BDC. FIG. 3A is a pXRD of Hf-BDC, Hf-BDC-PEG, T@Hf-BDC-PEG, B@Hf-BDC-PEG, and TB@Hf-BDC-PEG. FIG. 3C shows FTIR analysis of Hf-BDC, Hf-BDC-PEG, TB@Hf-BDC-PEG, and neat DSPE-PEG. FIG. 3D is a plot of DLS measurements and FIG. 3E shows zeta potential of Hf-BDC, Hf-BDC-PEG, and TB@Hf-BDC-PEG nMOFs in water. FIG. 3F demonstrates evaluation of colloidal stability of Hf-BDC-PEG in water, PBS, and media at 1, 4, and 7 days as measured by z-avg values.

FIGS. 4A and 4B show drug release profiles for (4A) talazoparib and (4B) buparlisib at pH5.5 and 7.4 (PBS, 25° C.). FIGS. 4C and 4D demonstrate enhancement of reactive oxygen species. (4C) Singlet oxygen sensor green (SOSG) and (4D) APF fluorescence assays for detection of 1O2 and .OH in the presence of Hf-BDC-PEG at increasing radiation doses as compared to control (water). * denotes statistically significant difference (***p<0.001, and **** p<0.0001).

FIGS. 5A-5C show in vitro cell viability of drug toxicity curves for Hf-BDC-PEG and TB@Hf-BDC-PEG in 4T1 cells (5A). 4T1 cells evaluating the synergetic effects of RT and (5B) Hf-BDC-PEG and (5C) TB@Hf-BDC-PEG.

FIGS. 6A and 6B are results of quantification of images of γH2AX foci in 4T1 cells treated with no drug (control), free TB, Hf-BDC-PEG, or TB@Hf-BDC-PEG and unirradiated or irradiated. Nuclei were stained for DAPI and γH2AX. FIG. 6A shows average number of γH2AX foci per cell and FIG. 6B shows percentage of cells expressing >5 γH2AX foci for unirradiated and irradiated samples 24 hours after radiation. Asterisk * denotes statistically significant difference (****p<0.0001 and ***p<0.001).

FIG. 7A shows results of a clonogenic assay for evaluation of radiosensitization upon X-ray irradiation on 4T1 cells and FIG. 7B is a table of radiation enhancement ratios (RERs) as determined by the survival fractions (SFs) for free TB, Hf-BDC-PEG, and TB@Hf-BDC-PEG at increasing radiation doses. * denotes statistically significant difference (*p<0.05, **p<0.01).

FIGS. 8A-8D demonstrate in vivo evaluation of acute and chronic toxicity of unloaded Hf-BDC-PEG nMOF. Blood clinical chemistry markers of liver and kidney function for PBS, 10 mg/kg (LD), and 55 mg/kg (HD) of Hf-BDC-PEG for (8A) acute (1 day) and (8B) chronic toxicity (30 days.). In vivo evaluation of biodistribution and clearance of Hf-BDC-PEG for 10 mg/kg (LD), and 55 mg/kg (HD) (8C) 10 mg/kg (LD) and (8D) 55 mg/kg (HD).

FIGS. 9A-9B show results of an in vivo tumor growth and treatment efficacy study using exemplary compositions. FIG. 9A shows tumor growth curves of in mice when treated with different therapeutic regimens, FIG. 9B shows relative tumor volumes; * Represent p<0.05.

FIG. 10A shows cell viability curves of TMZ and TMZ:Tal at a 30:1 ratio graphed against TMZ concentration. FIG. 10B shows cell viability curves of Tal and TMZ:Tal at a 30:1 ratio graphed against Tal concentration FIG. 10C shows Combination Index plot demonstrating synergy for various combinations of TMZ:Tal (30:1) utilizing Fa values between 0.1 and 0.9 from A and B.

FIG. 11A depicts representative nanoparticle coatings symbols utilized throughout study (top), potential (bottom right) and size by number (bottom left) measurements for the various nMOF coatings and drug loading FIG. 11B is a TEM image of Hf=BDC (top) and Hf-BDC-Fuco (bottom). Scale bar represents 200 nm in the main image and 20 nm in the inset image. FIGS. 11C-11F depict in vivo pilot study to determine optimal nMOF dosing regimen before incorporating radiation. FIG. 11C is a schematic representation of the three treatment schedules utilized for proof of concept study. FIG. 11D shows weight measurements over course of injections to evaluate treatment tolerability. FIG. 11E shows tumor growth curves for mice treated with various dosing schemes. FIG. 11F is a Kaplan-Meier survival curve to evaluate treatment efficacy. Asterisk (*) represent statistically significant difference (p<0.05) between the control (Hf-BDC-Fuco) and the 2×3 and 2×5 day treatment regimens analyzed by two-way ANOVA.

FIG. 12 is quantification of fold-change in P-selectin expression as a fraction of total tissue for various treatments; when compared to no radiation, the data demonstrate increase in P-selectin expression with respect to ionizing radiation (IR) dose and time after IR.

FIGS. 13A and 13B are graphs of average pixel intensity of multiple images for CT26.wt (13A) and bEnd.3 (13B) cells treated with uncoated, dextran coated, or fucoidan coated nMOFs with or without 2 gray IR. Asterisk (*) represents statistically significant difference (p<0.05) analyzed by two-way ANOVA with multiple comparisons.

FIG. 14A is a graph of metabolic-based viability of cells treated with fucoidan or Hf-BDC-Fucoidan (with or without radiation) across a range of doses. The concentration of free fucoidan is matched to the concentration of fucoidan on the nanoparticle surface. FIG. 14B depicts experimental plan for experiments results of which are shown in FIGS. 14C and 14D, demonstrating that nMOF concentration was kept constant across all wells (15 μg/mL) while dual-drug loaded exemplary nMOF was spiked into wells. In vitro viability for CT26.wt cells treated with a constant concentration of Hf-BDC and three coating conditions (uncoated, dextran coated, and fucoidan coated) with or without 2 gray and a TT concentration of 0.3 μM (14C) or 9.6 μM (14D). Asterisks (*) represent statistically significant difference (p<0.05) analyzed by one-ANOVA. FIG. 14E is a graph of a clonogenic assay investigating the radiosensitizing potential of various Hf-BDC-Fucoidan loaded particles. FIG. 14E shoes Radiation Enhancement Ratio values for all treatments in FIG. 14E compared to no treatment at a given RT dose.

FIG. 15A is a schematic representation of treatment schedule for efficacy study. FIG. 15B shows tumor growth curves for mice treated with various agents. Asterisks (*) represent statistically significant difference (p<0.05) between Hf-BDC-Fuco and all other groups as analyzed by two-way ANOVA with multiple comparisons. FIG. 15C is a Kaplan-Meier survival curve to evaluated treatment efficacy. Asterisks (*) represent statistically significant difference (p<0.05) between Hf-BDC-Fuco and exemplary composition TT@Hf-BDC-Fuco+RT as analyzed by Log-rank (Mantel-Cox) test. FIG. 15D shows tumor density values for each treatment group determined by dividing final tumor mass by final tumor volume. FIG. 15E shows tumor growth rate values for each treatment group as determined using base-10 logarithmic scale. Asterisks (*) represent statistically significant difference (p<0.05) between Hf-BDC-Fuco and all other groups as analyzed by one-way ANOVA with multiple comparisons. FIG. 15F shows quantification of hypoxic/necrotic regions as a fraction of whole tumor slice.

FIG. 16A shows mouse body weight evaluation to evaluate treatment-associated toxicity. FIG. 16B shows complete blood count for mice in each treatment group to evaluate hematologic toxicity. FIGS. 16C and 16D show clinical chemistry evaluation of Liver (ALT, AST, Total Protein) and Kidney (BUN, A/G) function, respectively. Asterisks (*) represent statistically significant difference (p<0.05).

### DETAILED DESCRIPTION

Multimodal radiation therapy (MMRT) combining cytotoxic, targeted, and immunotherapies with radiation is an extremely valuable treatment option. However, despite its efficacy, MMRT is not universally utilized in part due to the increased nonspecific toxicity elicited by these combinations. Nanoparticles present a unique opportunity to overcome the issues of compounded toxicity by preferentially delivering drug to the tumor and microenvironment where the maximal dose of radiation will be delivered. Nanoparticles utilized in chemoradiation can typically be categorized as inorganic-based radiation dose enhancing nanoparticles or as organic-based radiation sensitizer carriers.

The present disclosure provides chemotherapeutic compositions comprising a hybrid material comprising nanosized metal-organic-framework (nMOFs). The present disclosure further provides compositions and methods for inflammation-targeted delivery of MMRT agents to tumors and their use as cancer therapeutic agents.

The compositions of the disclosure can enhance radiation through physical means and deliver hydrophobic chemotherapeutics, e.g., those that can act as sensitizers of radiation. In some embodiments, this platform relies on passive methods of accumulation. In some embodiments, the compositions of the disclosure have been engineered to target cancer- and tumor microenvironment-specific proteins of interest.

Thus, in one aspect, the disclosure provides a composition comprising a hybrid material comprising nanosized metal-organic-framework (nMOF) and two or more therapeutic agents selected from the group consisting of DNA-targeting chemotherapeutic agents, DNA Damage Repair (DDR) inhibitors, and a combination thereof. When used in combination with radiotherapy, the agents of the compositions can act as radiosensitizers.

In some embodiments, the therapeutic agents comprise a combination of one or more DNA-targeting chemotherapeutic agents and one or more DNA Damage Repair (DDR) inhibitors. In some embodiments, the therapeutic agents comprise a combination of two or more DNA Damage Repair (DDR) inhibitors and optionally a DNA-targeting chemotherapeutic agent. In some embodiments, the therapeutic agents comprise a combination of two or more DNA Damage Repair (DDR) inhibitors.

The compositions of the disclosure comprise nanosized metal-organic frameworks (nMOFs) which are a class of hybrid materials self-assembled from organic bridging ligands and metal ion/cluster connecting points. Any suitable nanosized metal-organic-framework (nMOF) can be used in the compositions of the disclosure. In some embodiments, nMOF are high Z element nMOFs. In some embodiments, the nMOF are Group 4 metal-based metal-organic frameworks, (M^{IV}-nMOFs), including Ti-, Zr-, and Hf-based nMOFs. In some embodiments, the nanosized metal-organic-framework (nMOF) comprises Hf and 1,4-benzenedicarboxylic acid (Hf-BDC). In some embodiments, the nMOF is Zr or Hf terephthalate nMOF, e.g., MOF UiO-66.

In some embodiments, the nanosized metal-organic-framework (nMOF) can be coated with a PEG or PEGylated. In some embodiments, PEG chains can enhance stability of the nMOF at physiological pH (e.g., pH 7.4), e.g., toward phosphates, and thus overcome the “burst release” phenomenon by blocking interaction with the exterior of the nanoparticles, whereas at lower pH (e.g., pH 5.5), release of the agent associated with the nMOF can be achieved. PEGylation can enhance cellular internalization of PEGylated nMOF by potentially escaping lysosomal degradation through enhanced caveolae-mediated uptake. Methods of PEGylation by covalent attachment of PEG derivatives to groups present in nMOF ligands are known in the art. For example, some of such methods are disclosed in Abánades Lázaro I. et al., Selective Surface PEGylation of UiO-66 Nanoparticles for Enhanced Stability, Cell Uptake, and pH-Responsive Drug Delivery. Chem. 2017; 2(4): 561-578, the disclosure of which is incorporated herein by reference. In some embodiments, PEGylation can be achieved by non-covalent coating of nMOF with a suitable derivative of PEG, such as a lipid comprising PEG, for example, 1,2-distearoyl-sn-glycero-3-phosphoethanolamine-poly (ethylene glycol) (DSPE-PEG).

In some embodiments, the nanosized metal-organic-framework (nMOF) is coated with an agent that has affinity to a cell surface protein upregulated under inflammatory conditions, such as inflammation caused by ionizing radiation (IR). The inventors discovered that coating the nMOFs with a targeting polysaccharide, e.g., fucoidan, leads to increased nMOF uptake in irradiated cells. This enhanced uptake in turn leads to enhanced IR toxicity when combined with administration of fucoidan-coated nMOF of the disclosure.

In some embodiments, the nanosized metal-organic-framework (nMOF) is coated with a polysaccharide. In some embodiments, the polysaccharide has affinity for P-Selectin. In some embodiments, the compositions disclosed herein include nMOF (such as Hf-BDC nMOFs) comprising a coating of a naturally derived polysaccharide, e.g., fucoidan or P-Selectin glycoprotein ligand 1. In some embodiments, such coating is facilitated by an electrostatic interaction between the positively charged Hf-BDC surface and the negatively charged polysaccharide, e.g., fucoidan. Fucoidan is a sulfated fucose molecule, wherein the sulfate groups provide the basis for the negative charge of the molecule. Fucoidan is a known substrate for the cellular adhesion molecule P-selectin, which is translocated in response to various stimuli, such as ionizing radiation, that induce inflammation.

Typically, the agents that can be included in the compositions of the disclosure are hydrophobic agents. In some embodiments, the one or more DNA-targeting chemotherapeutic agents is hydrophobic. In some embodiments, the one or more one or more DNA Damage Repair (DDR) inhibitors is hydrophobic.

In some embodiments, the compositions comprise one or more DNA-targeting chemotherapeutic agents, such as a platinum agent, a triazene, a nitrosourea, an alkylating hexitol, or a nitrogen mustard. In some embodiments, the one or more DNA-targeting chemotherapeutic agents is triazene agent, such as dacarbazine, procarbazine, or temozolomide.

In some embodiments, the one or more one or more DNA Damage Repair (DDR) inhibitors is a tyrosine kinase inhibitor or a poly (ADP-ribose) polymerase (PARP) inhibitor. In some embodiments, the one or more one or more DNA Damage Repair (DDR) inhibitors is rucaparib, talazoparib, niraparib, or olaparib.

In some embodiments, the compositions comprise talazoparib. In some embodiments, the compositions comprise talazoparib and buparlisib. In some embodiments, the compositions comprise talazoparib and temozolomide.

In some embodiments, the compositions comprise P-selectin-targeted nMOF which are able to deliver a combination of a PARP inhibitor and a DNA alkylating agent to enhance the therapeutic efficacy of radiation therapy. In some embodiments, each of the PARP inhibitor and DNA-targeting agents is loaded in the compositions at the concentration of about 100 μM and about 700 μM, respectively; about 300 μM and about 1000 μM, respectively; about 400 μM and about 2000 μM, respectively; about 400 μM and about 4000 μM, respectively; or about 1000 μM and 7000 μM, respectively. In some embodiments, the drug loading efficiency is about 75% or greater, about 80% or greater, about 85% or greater, about 90% or greater, about 95% or greater. High loading efficiencies (e.g., of about 75%-95%) and the resulting concentrations of such agents in the composition of the disclosure allow to advantageously provide a high local (e.g., intratumoral) concentration of the agent after administration of ionizing radiation to the tumor, for example, when the radiation dose is a dose sufficient to generate inflammatory response within the tumor. In some embodiments, when more than one agent is present, the loading of the other one or more chemotherapeutic agent can depend on the concentration of the first agent. In some embodiments, the concentrations of the agents are chosen to maximize their synergistic effect with the radiation treatment. In some embodiments, encapsulation of the hydrophobic agents in the nMOF as disclosed herein decreases each drug's concentration required for efficacy (e.g., IC₅₀), for example, by about at least 10%, at least 20%, at least 30%, at least 40%, at least 50%, at least 60%, at least 70%, at least 80%, or at least 90%. In some embodiments, the concentration required for efficacy is decreased to-fold, five-fold, or ten-fold. In some embodiments, the two or more chemotherapeutic agents demonstrate synergistic effect with each other and/or radiation.

The nMOF particles of the composition of the disclosure have sub-micron sizes. In some embodiments, the particles have z-average sizes as determined by dynamic light scattering (DLS) of about 40 nm, about 50 nm, about 60 nm, 70 nm, about 80 nm, about 90 nm, about 100 nm, about 110 nm, about 120 nm, about 130 nm before coating and/or drug loading. In some embodiments, the particles have z-average sizes as determined by dynamic light scattering (DLS) of about 40 nm, about 50 nm, about 60 nm, 70 nm, about 80 nm, about 90 nm, about 100 nm, about 110 nm, about 120 nm, about 130 nm after coating and/or drug loading.

In some embodiments, exemplary fucoidan-targeted compositions disclosed herein can be prepared as follows. nMOF can be suspended at a desired concentration in appropriate aqueous media (e.g., phosphate buffered saline (PBS) or water) to form a dispersion. Once the nMOF particles are fully dispersed, chemotherapeutic agents (e.g., Talazoparib and Temozolomide) as a solution in a suitable solvent (e.g., dimethyl sulfoxide (DMSO)) at a desired concentration can be added to the nMOF dispersion. After drug loading, a solution of a suitable polysaccharide (e.g, fucoidan) can be added, and after completion of the formation of fucoidan-coated, drug-loaded nMOF compositions, drug loading efficiency can be quantified (e.g., by analysis of the supernatants to evaluate the decrease in the drug amounts and thus quantify the drug loading). Using the methods described above, high drug loading efficiencies and concentrations in the compositions can be achieved, which can allow maximization of the therapeutic index of the compositions of the disclosure.

In another aspect, provided herein is a method of treatment of cancer in a subject, comprising administering to a subject in need thereof an effective therapeutic dose of a composition of the disclosure.

In some embodiments, the compositions of the disclosure can be administered intravenously to oncology patients who are undergoing a course of radiotherapy. In some embodiments, the methods disclosed herein further comprise administering a therapeutically effective dose of ionizing radiation (IR) to the patient. Administration of a therapeutically effective dose of radiation to the patient can be done prior to, concurrently with, or after the administration of the composition. In some embodiments, the therapeutically effective dose of radiation is a dose that results in increased expression of a cell surface protein upregulated under inflammatory conditions. In some embodiments, the dose that results in increased expression of a cell surface protein upregulated under inflammatory conditions is lower than the dose required for tumor treatment by radiation alone. In some embodiments, the therapeutically effective dose of radiation is a dose that results in increased expression of P-Selectin in the tumor treated with the radiation compared to the pre-treatment expression. Methods of determining expression of a protein of interest are known in the art.

Cancers that can be treated using the methods and compositions of the disclosure include colorectal cancer, breast cancer, head and neck cancer, esophageal cancer, gastric cancer, lung cancer, pancreatic cancer, gastric cancer, bladder cancer, cervical cancer, brain tumors, such as glioblastomas, and the like. In some embodiments, the cancer is a solid tumor. In some embodiments, administration of the compositions disclosed herein inhibits the development of a hypoxic/necrotic core in the tumor. In some embodiments, the compositions cause apoptotic death of the tumor cells. In some embodiments, administration of therapeutically effective doses of the compositions of the disclosure do not result in systemic toxicity.

Any suitable method of administration can be used to deliver the compositions of the disclosure. In some embodiments, the composition is administered intravenously or intratumorally, e.g., by injection. In some embodiments, the compositions of the disclosure are delivered with one or more pharmaceutically acceptable carriers, e.g., carriers and formulations adapted for intravenous or intratumoral injection.

In another aspect, provided herein is a pharmaceutical composition comprising a composition of the disclosure.

In some embodiments, due to the ability of fucoidan to target P-selectin which is upregulated under inflammatory conditions, the compositions can be used to carry therapeutics selectively to tissues in patients with inflammatory disorders such as rheumatoid arthritis, etc.

Unless specifically defined herein, all terms used herein have the same meaning as they would to one skilled in the art of the present invention. The use of the term “or” in the claims is used to mean “and/or” unless explicitly indicated to refer to alternatives only or the alternatives are mutually exclusive, although the disclosure supports a definition that refers to only alternatives and “and/or.” The words “a” and “an,” when used in conjunction with the word “comprising” in the claims or specification, denote one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element. Additionally, the words “herein,” “above,” and “below,” and words of similar import, when used in this application, shall refer to this application as a whole and not to any particular portions of the application. As used herein, the term “about” includes ±10% of the stated value.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these components etc. may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

All publications cited herein and the subject matter for which they are cited are hereby specifically incorporated by reference in their entireties.

The following examples are provided to illustrate certain particular features and/or embodiments of the disclosure. The examples should not be construed to limit the disclosure to the particular features or embodiments described.

### Examples

### A. Co-delivery of PARP and PI3K Inhibitors by Exemplary Nanoscale Metal-Organic Framework Compositions for Enhanced Tumor Chemoradiation

This example illustrates preparation and characterization of an exemplary nMOF composition comprising a PARP inhibitor and a PI3K inhibitor and demonstrates its use in combination with radiotherapy in treatment of cancers.

Materials.

All materials were used without further purification. Powder X-ray diffraction (pXRD) patterns were performed using a Rigaku Ultima IV X-ray diffraction system in parallel beam geometry with a graphite monochromatized Cu K alpha radiation. Samples were prepared by dispersing dry powders in methanol using ultrasonication and drop casting the solutions onto silicon wafers that were heated to 40° C. Dynamic light scattering (DLS) was performed using a Malvern Nano ZSP (Malvern Panalytical, Malvern, UK) to determine the size, zeta potential, and polydispersity. Transmission electron microscopy (TEM) was performed using a Technai F-20 TEM operating at an accelerating voltage of 4200 eV. Dry powder samples were suspended in methanol at approximate concentrations of 5 mg/mL, ultrasonicated for 5 minutes, then drop cast onto TEM sample grids (Ted Pella Inc., Product #01824). The grids were dried at room temperature for 20 minutes, then 60° C. for 10 minutes before analysis. Hafnium concentrations were determined using an Agilent 7900 inductively coupled plasma mass spectrometer (ICP-MS). Helium was used as a diffusion gas. Samples were digested in concentrated HNO3 overnight. Samples were than diluted in 2% HNO₃ aqueous solutions. A calibration curve was produced using a hafnium standard acquired from RICCA. All standards were subjected to the same digestion process as the samples. Drug loading was quantified using a Shimadzu SPD-20A high performance liquid chromatography (HPLC) instrument (Torrance, Calif., USA) equipped with UV/Vis detector and an Agilent Zorbax Rapid Resolution SBC-18 column (4.6×100 mm, 3.5 μm; Santa Clara, Calif., USA).

Synthesis of Exemplary Hf-BDC Nanoparticles.

Synthesis of Hf-BDC nMOF was carried out using a modified method. In brief, HfCl₄ (1.5 g, 4.6 mmol), BDC (1.07 g, 6.4 mmol), and acetic acid (44 ml) were added to a round bottom flask and dissolved in DMF to which H₂O (7.5 mL) was then added. The resulting solution was than heated in an oil bath under stirring at 120° C. for 15-20 minutes followed by cooling to room temperature. The resulting white Hf-BDC nMOFs were than collected via centrifugation and washed three times with DMF to remove unreacted materials. Following the nMOFs were further purified with ethanol washing several times to remove residual DMF. The nMOFs were then dried overnight to remove residual solvents.

PEGylation of Hf-BDC nMOFs.

DSPE-PEG2000 was dissolved in MeOH (2.5 mg/mL) and added at a Hf-BDC:DSPE-PEG2000 2:1 ratio (w/w) of nMOF dispersed in MeOH. The resulting mixture was then vortexed briefly and concentrated with a nitrogen purge. After evaporation of the solvent, a dry film of nanoparticles remained to which distilled water was added, followed by brief vortexing and sonication to obtain Hf-BDC-PEG. The nMOF suspension was then centrifuged to remove excess polymer and redispersed in Millipore water. PEGylation of Hf-BDC was confirmed by DLS to evaluate change in particle size and zeta potential. Influence of PEGylation on MOF stability was investigated by dispersing Hf-BDC-PEG in water, PBS, and cell media and performing DLS measurements over the course of one week (1, 3, and 7 days) to analyze the z-avg and PDI.

Drug Loading/Drug Release Studies of Talazoparib and Buparlisib-Loaded nMOFs (TB@Hf-BDC-PEG).

Talazoparib and buparlisib were loaded into the Hf-BDC-PEG nMOFs by directly adding aliquots of the dissolved drugs (10 mg/mL in DMSO) at the desired concentrations into distilled water (2 mL) with the Hf-BDC-PEG nMOFs (approximately 5.0 mg) and stirred overnight. Unloaded drug(s) were removed by centrifugation and the supernatant was collected and quantified on HPLC to determine the concentration of unloaded talazoparib and buparlisib. A calibration curve for talazoparib and buparlisib was obtained using standard solutions at different concentrations and the curve was fitted with linear regression. Encapsulation efficiency was determined based on the ratio of the amount of drug loaded in Hf-BDC-PEG to the total amount added. The resulting nMOFs were dried under vacuum and stored until use. The influence of pH on drug release of talazoparib and buparlisib from the Hf-BDC-PEG nMOF was investigated using dialysis. TB@Hf-MOF (1.6 mg, 500 μL) was loaded into Slide-A-Lyzer MINI dialysis microtubes with a molecular weight cutoff of 2,000. The loaded samples were dialyzed against 12 mL of buffer (pH 5.5 or 7.4 PBS) with gentle stirring under ambient conditions. At designated time points of 0, 1, 2, 4, 24, 48, 72, 168, and 336 hours, sink samples (6 mL) were collected and replaced with fresh solution. Talazoparib and buparlisib concentrations were determined via HPLC, and drug release profiles were plotted as cumulative % of total drug release vs. time. All drug release measurements were performed in triplicate.

Detection of Singlet Oxygen Formation with SOSG Assay.

The ability of the Hf-BDC-PEG nMOF to generate singlet oxygen upon irradiation was investigated using the singlet oxygen sensor green (SOSG) assay (Thermo Fischer Scientific, USA). In a 96 well-plate, 150 μL solutions were added containing either water (control), Hf-BDC (5 μg/mL), or Hf-BDC-PEG (5 μg/ml) in triplicate. SOSG was then added to all wells to give a final concentration of 10 uM and irradiated with X-rays (CellRad, Faxitron, 130 kV, 5 mA, ˜1.2 Gy/min) at 0, 2, 4, 6, 8, 10, 12, and 14 Gy. The fluorescence signal (488/525 nm) was immediately read on an Infinite M200 Pro plate reader (Tecan US Inc, Morrisville, N.C., USA).

Detection of Hydroxyl Radical Formation with APF Assay.

Hydroxyl radical formation of the Hf-BDC-PEG nMOF was evaluated using the aminophenylfluorescein (APF) assay (ThermoFisher, USA). In the presence of hydroxyl radicals APF (nonfluorescent) reacts to give off a bright green fluorescence (ex/em 490/515 nm). In a 96 well-plate, 150 μL, solutions were added containing either water (control), Hf-BDC (200 ug/mL), or Hf-BDC-PEG (200 ug/ml) in triplicate. APF was then added to all wells to give a final concentration of 8 uM and irradiated with X-rays (CellRad, Faxitron, 130 kV, 5 mA, ˜1.2 Gy/min) at 0, 2, 4, 6, 8, 10, 12 and 14 Gy. The fluorescence signal (490/520 nm) was immediately read on an Infinite M200 Pro plate reader.

In Vitro Cytotoxicity Studies.

Murine mammary (4T1) cells were purchased from American Type Culture Collection (ATCC) and seeded at 2.0×103 cells per well in 96 well plates. The cells were allowed to settle and grow for approximately 24 hours prior to dosing. A dosing plate was prepared at maximum concentrations of 250 μg/mL for Hf-BDC-PEG and TB@Hf-BDC-PEG nMOFs in media. The maximum concentration was diluted with media by one-quarter eight times, giving nine dilutions. Those dilutions were added to the plates in triplicate (technical replicates), with 200 μL per well. The drug was incubated for 72 hours at 37° C. at which point wells were washed twice with sterile PBS before adding 10% alamar blue in media. The plates were read on a Infinite M200 Pro plate reader (Tecan US Inc, Morrisville, N.C., USA) after two hours of incubation at 37° C., with excitation 530 nm and emission 590 nm. Cell viability results were normalized to untreated cells (control) and IC₅₀ values were determined using GraphPad Prism 7 by fitting the data to either a three parameter or four parameter dose response variable slope model depending on the best fit. Combination indexes were calculated using CompuSyn (ComboSyn Inc., Paramus, N.J., USA.) All experiments were performed in triplicate (biologic replicates). The above was also performed in HEPG2 cells and NIH3T3 fibroblasts, except these cells were each plated at a higher density (10×103 cells per well) and cells were treated only with Hf-BDC-PEG nMOF solutions diluted by half instead of quarter.

Cy 7.5@Hf-BDC-PEG Cell Uptake.

4T1 cells were seeded at approximately 3.0×103 cells per well in a 96 well plate and allowed to adhere over 24 hours. Hf-BDC-PEG nMOFs were loaded with luminprobe cyanine 7.5 carboxylic acid dye (Cy7.5). Cy 7.5@Hf-BDC-PEG was diluted (31.25 μg/mL) and Cy7.5 in media (340 ng/mL). The diluted nanoparticles (100 μL) were added to the cells in triplicate at time points for 24, 6, 4.5, 3, 2, 1, 0.5, 0.25, and 0 hours of incubation. After 24 hours, the media was removed, and all wells were washed with PBS (100 μL) two times. The cells were then fixed with 10% phosphate buffered formalin, followed by a final PBS wash. DAPI dilactate and Alexa Fluor 488 Phalloidin (D3571 & A12379, Thermo Fisher Scientific) stain were incubated with cells for 10 minutes to visualize nuclei and actin respectively. Fluorescent images were taken at 40× magnification on an EVOS FL Auto Imaging System for all wells. The overall fluorescence intensity of the Cy7.5 channel for each image was quantified using FIJI representing the amount of nanoparticle uptake.

Hf-BDC-PEG Cell Uptake.

4T1 cells were seeded at 1.0×104 cells per well in a two 6-well plate to represent technical replicates and allowed to adhere for 24 hours. 40 μg/mL Hf-BDC-PEG nMOFs were incubated with cells for 24, 6, 4, 2, 0 h, an additional well received no treatment and acted as the control. The incubated cells were washed with PBS 3 times, trypsinized, and resuspended in PBS (2 mL) and counted. Cells were collected by centrifugation at 500 rpm and the cell pellet was dispersed in PBS (100 μL) and transferred to a digestion tube to which concentrated HNO₃ (100 μL) was added. Samples were then heated to 90° C. overnight followed by the addition of 2% HNO₃ (5 mL). Samples were mixed thoroughly and transferred to 15 mL conical tubes and adjusted to a total volume of 10 mL and analyzed by ICP-MS to determine the total concentration of hafnium present in the cells.

Immunofluorescent γH2AX DNA Damage Assay.

4T1 cells were seeded at 8.0×104 cells per well in four gelatin-coated 4-well chamber slides and allowed to adhere over 24 hours. One well per plate were dosed with each treatment: media alone, Hf-BDC-PEG at 31.25 μg/mL MOF, free drug (0.2 μM talazoparib and 0.25 μM buparlisib), and TB@Hf-BDC-PEG nMOF (0.2 μM talazoparib, 0.25 μM buparlisib). The drug was incubated for 5 hours at which point two of the four plates were irradiated with 4 Gy. 15 minutes after irradiation, one irradiated and one unirradiated plate was washed with PBS and fixed. Similarly, 24 hours after irradiation the process was repeated with the remaining 2 plates. Cells were incubated with 5% Goat Serum and 0.3% Triton X-100 in PBS for one hour to block and permeabilize the cells.

The cells were then incubated with a rabbit anti-Phospho-Histone Gamma-H2AX (Ser139) Antibody (#2577, Cell Signaling Technologies) at a 1:800 dilution in antibody dilution buffer (1% BSA and 0.3% Triton X-100 in PBS) for one hour at room temperature. Following the primary antibody, cells were washed and incubated with a goat anti-rabbit IgG H&L (Cy5) antibody (6564, Abcam) at a 1:600 dilution in antibody dilution buffer at room temperature in addition to DAPI dilactate and Alexa Fluor 488 Phalloidin. Cells were once more washed with PBS and imaged using an EVOS FL Auto microscope (BRCA proficient 4T1 40×, BRCA deficient MDA-MB-436 20× magnification). Using 40×EVOS images (>150 cells/treatment group in various images), DNA damage quantification was automated by cell profiler overlaying the DAPI stained nucleus with the Cy 5 stained γH2AX foci for a foci/cell count. To be counted, a focus had to be greater than 7 pixels.

Clonogenic Assay.

4T1 cells were seeded in four T75 flasks at approximately 1.5×106 cells each and allowed to adhere and grow for 24 hours prior to dosing. One of the flasks was left untreated, and the other three flasks were dosed with free drug (2.3 μM talazoparib and 0.31 μM buparlisib), 31.25 μg/mL of Hf-BDC-PEG, TB@Hf-BDC-PEG (2.3 μM talazoparib, 0.31 μM buparlisib). Sixteen hours after treatment, the media containing treatment was removed and the flasks were cultured and each was plated into 3 wells of five 12-well plates (1 plate each for 0, 2, 4, 6, and 8 Gy). The cell plating numbers ranged from 300 cells/well for no treatment to 9600 cells/well for TB@Hf-BDC-PEG with 8 Gy. The cells were irradiated (CellRad, Faxitron, 130 kV, 5 mA, 0.5 mm aluminum filter, ˜1.2 Gy/min) immediately after plating in the 12-well plates. The cells were allowed to grow for 7 days, then the plates were rinsed once with PBS, fixed with 10% formaldehyde, washed once more with PBS, and stained with crystal violet. The colonies of greater than 50 cells were counted. The survival fraction was calculated by dividing the plating efficiency of each treatment by the untreated control for that treatment.

Hemolysis Assay.

The hemolysis properties of the Hf-BDC-PEG nMOF on red blood cells was investigated using a spectroscopic method. In brief, fresh blood samples were obtained from healthy, tumor-free BALB/c mice via cardia puncture and collected into lithium heparin tubes. Samples were than centrifuged down to separate the RBCs from the blood serum at 500 rpm for 10 min. Blood samples were washed with PBS two times, then diluted to 2 mL using PBS. Following, the red blood cell suspension (200 μL) was added to Hf-BDC-PEG (200 μL) nMOFs at varying concentrations (1000, 500, 250, 125, 62.5, 31.25, 15.63, 7.81, 3.91 and 1.95 μg/mL). Additionally, the red blood cell suspension (200 μL) was added to either PBS (200 μL) or triton-X (lyses red blood cells) to serve as negative and positive controls, respectively. After the samples were incubated in a 37° C. water bath for 3 hours they were centrifuged at 500 rpm for 10 min after which supernatants were transferred to a 96-well plate. The absorbance signal was read at 540 nm on an Infinite M200 Pro plate reader. The percentage of hemolysis was then calculated using the following equation:

% Hemolysis=(Aₛₐₘₚₗₑ−A_{control(neg)}/(A_{control(pos)}A_{control(neg)})

In Vivo Studies.

All animal studies were approved by and conducted following the guidelines of the Institutional Animal Care and Use Committee (IACUC) of Oregon Health and Science University. For all studies, mice were anesthetized using 2-3% isoflurane (Piramal Enterprises Limited, Telangana, India). Safety and Toxicity of Hf-BDC-PEG nMOFs. To study the potential acute and chronic toxicity of the unloaded nMOFs, healthy, tumor-free female BALB/c mice (5 weeks) were intravenously injected with either PBS, 10 mg/kg (low dose) Hf-BDC-PEG, or 55 mg/kg (high dose) Hf-BD).C-PEG. At 1 (acute) or 30 (chronic) days after treatment, mice were anesthetized, and blood was collected into 1 mL lithium heparin coated tubes (Grenier Bio-One, Kremsmünster, Austria) via cardia puncture for a mini chemistry panel test. Additionally, the heart, lungs, liver, spleen, and kidneys were collected at the respective time point. Following collection, all organs were weighed, split, and re-weighed. Approximately half of each organ was stored in either formaldehyde for tumor histology or in cryovials for biodistribution. All blood samples were stored at 4° C. prior to analysis by IDEXX Laboratories (Portland, Oreg., USA).

Biodistribution and Clearance of Hf-BDC-PEG nMOFs.

To evaluate Hf-BDC-PEG biodistribution weighed were homogenized in Millipore water at 5× their weight. Following homogenization, 100 μL aliquots were digested in concentrated HNO₃ at a 1:1 ratio overnight. After cooling to room temperature, each of the solutions was diluted to 10 mL with 2% HNO₃ and analyzed by ICP-MS to determine the total concentration of hafnium in each of the organs. The % injected dose per gram (ID/g) was then calculated based on the following equations:

$$\% ID = \frac{ng {Hf}_{organ}}{ng {Hf}_{total}} \times 100 , \% \frac{ID}{g} = \frac{\% ID}{organ weight} \times 100$$

Evaluation of In Vivo Tumor Growth Inhibition and Long-Term Toxicity Effects.

Subcutaneous 4T1 xenografts were established by injecting 50,000 cells in PBS into the right flanks of female, 5-week old BALB/c mice (Charles River Laboratories, Wilmington, Mass.). When tumors reached 50-150 mm3, mice were randomized into one of six treatment groups (n=5-6 per group). Treatment groups were as follows: (1) PBS (control), (2) Hf-BDC-PEG nMOF, (3) TB@Hf-BDC-PEG nMOF, (4) PBS+RT, (5) Hf-BDC-PEG+RT, (6) TB@Hf-BDC-PEG+RT. Mice were treated via tail vein injection (40 mg/kg, 3 mg/kg talazoparib, 0.4 mg/kg buparlisib) for 3 consecutive days starting on day 1 Mice in RT treatment groups were irradiated with 2 Gy on days 1, 2, and 3 for a total of 6 Gy. To prevent off-target radiation, only mice with tumors which were >1 mm from the spine were randomized into +RT groups. Radiation was administered (CellRad, Faxitron, 130 kV, 5 mA, 0.5 mm aluminum filter) selectively to tumors by shielding the mice with halfmoon cutout lead shields (Precision X-Ray, North Branford, Conn., USA). Body weights and average tumor diameter (½×length×width2) were recorded 3× weekly. All mice were sacrificed on Day 14 and the tumors were excised, weighed, and formalin fixed.

Tumor Histology.

Tumors from mice were collected, weighed, and fixed overnight in a 10% neutral buffered formalin solution at 4° C. Following initial fixing, tumors were placed in tissue cassettes and transferred to a 70% ethanol solution for further processing by the OHSU Histopathology Shared Resource Core. Tumor tissues were sliced and stained for hematoxylin and eosin (H&E), cleaved caspase-3 (Promega), and Ki67 (Cell Marque). All staining was performed at antibody dilutions recommended by the supplier.

Statistical Analysis.

Statistical analysis was performed using GraphPad Prism 7 software. Significant differences in the data were determined by Student's t test or ANOVA where p values less than 0.05 were considered significant.

Results

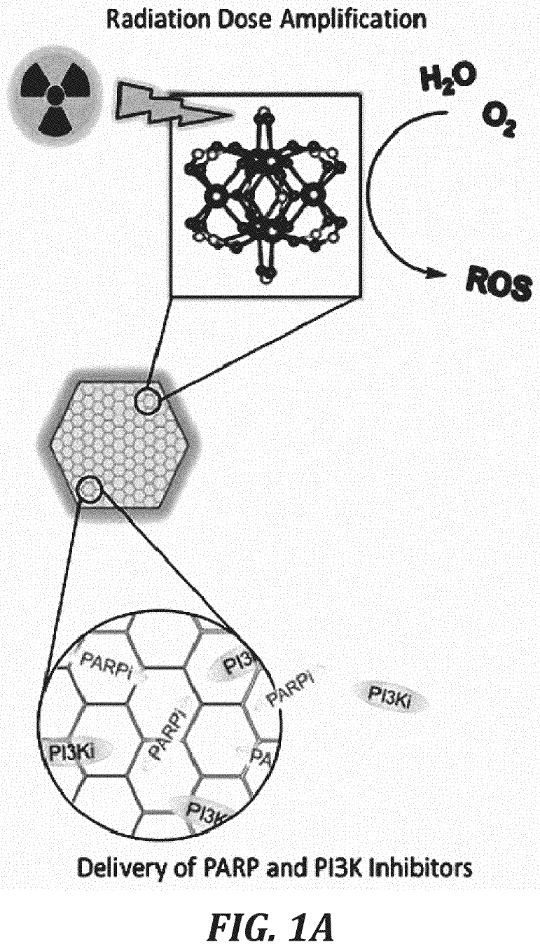

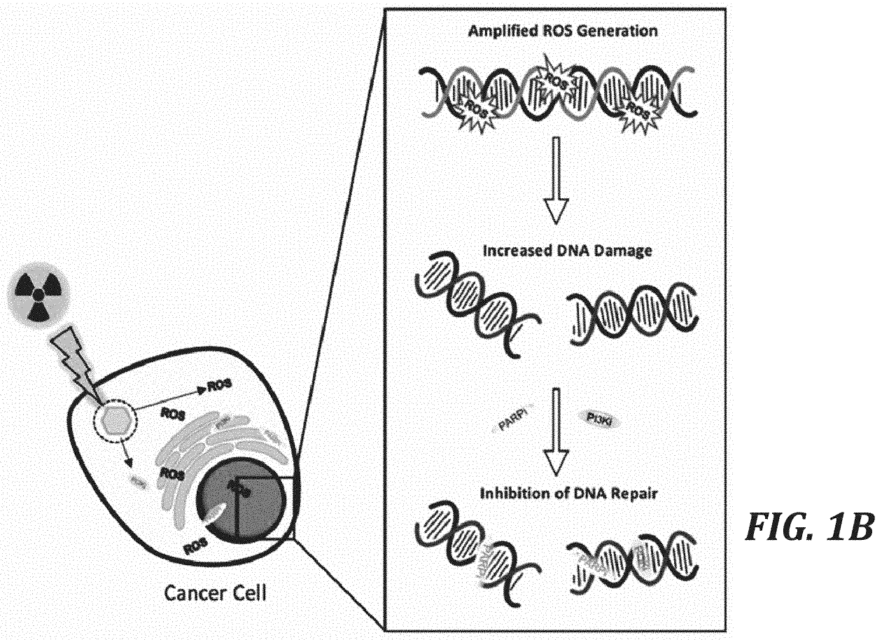

Metal-organic frameworks (MOFs) represent a class of hybrid, multi-dimensional materials derived from metal ions or clusters connected by organic ligands. This class of materials boasts advantageous characteristics such as; high porosity, tunability, and multifunctionality, rendering them useful for numerous applications. Recently, the development and evolution of nanoscale MOFs (nMOFs) has attracted significant interest for their utility as therapeutic platforms for oncology and other diseases. Compared to traditional inorganic-based nanoparticle platforms utilized for radiation dose enhancement, nMOFs offer several key advantages including increased loading capacities, biodegrability, and decreased toxicity. The ability of nMOFs to interact with external stimuli has become an area of active investigation. In particular, several reports have demonstrated the ability of Hafnium (HO-based nMOFs to generate increased levels of reactive oxygen species (ROS), both hydroxyl radicals (.OH) and singlet oxygen (¹O₂), upon irradiation for RT therapy. Increasing the amount and diffusion of ROS should improve radiation-local therapeutic efficacy since the interaction of ROS with biological molecules (lipids, DNA, etc.) is one of the primary mechanisms of radiation-induced cellular damage. As nMOFs are commonly composed of highly electron dense elements, there is also greater potential for local radiation interactions and increased dose deposition compared with that of tissue alone. At the same time, their high porosity facilitates efficient storage and transport of both hydrophilic and hydrophobic cargo. Thus, by exploiting these inherent material properties, nMOFs that stimulate stronger radiation enhancing effects and simultaneously deliver radiosensitizing drugs may be developed leading to improved therapeutic efficacy (FIG. 1). However, to date, these combined therapeutic routes remain relatively unexplored.

Mechanistically, RT induces DNA damage through the formation of highly reactive chemical species (i.e., ¹O₂, .OH), leading to the formation of both single-strand breaks (SSBs) and double-strand breaks (DSBs), the latter being far more lethal. Radiosensitizing chemotherapeutic approaches capitalize on this DNA damage by inhibiting post-radiation repair, thus synergizing with RT effects to prevent survival. Theoretically, in contrast to conventional chemotherapeutics, the maximal effects of DNA damage repair (DDR) inhibitors should be confined to areas with an increased degree of damage (i.e., radiated tissue). DDR is a complicated process which involves many proteins, all of which present unique options for targeted DDR inhibition. Of particular interest to this study, poly (ADP-ribose) polymerase (PARP) and phosphoinositide 3-kinase (PI3K) play fundamental roles in cellular processes including the repair of both SSB and DSBs. Studies have suggested PARP inhibition during RT can induce tumor growth inhibition with minimal effects on healthy tissue making it a desirable therapeutic option. Furthermore, various combinations of PARP and PI3K inhibitors have demonstrated synergy in endometrial, breast, ovarian, and prostate cancer models indicating the high degree of versatility that dual-administration exhibits. Similar to many small molecule inhibitors, the in vivo translation of PARP and PI3K inhibitors is hindered by their poor water solubility, limiting their therapeutic efficiency and eliciting a need for alternative delivery strategies. Toward this end, co-delivery of talazoparib (PARP inhibitor) and buparlisib (PI3K inhibitor) in a micellar formulation resulted in in vitro synergy and increased in vivo DNA damage and cell death during RT. However, in vivo administration of talazoparib and buparlisib did not result in enhanced survival rates compared to RT only. This highlights the complicated nature of chemoradiation and the necessary role of optimization in terms of variables such as the dose per injection, dosing sequence, and total radiation dose. Additionally, these materials were constrained by the inability of the micellular formulations to encapsulate high drug concentrations at non-toxic polymer concentrations. Moving forward, the incorporation of these hydrophobic DDR inhibitors into nMOFs, is a multi-pronged approach that enables increased payload delivery, enhanced radiation sensitivity, and the localized prevention of DNA repair thereafter.

The inventors evaluated a unique, synergistic treatment strategy for enhancing the therapeutic efficacy of RT via nMOF-mediated drug delivery and RT enhancement. Here, a nMOF (Hf-BDC) containing the high-Z element Hf and the ligand 1,4-benzenedicarboxylic acid (BDC) was synthesized and evaluated for its potential as a chemoradiation platform. To validate the therapeutic potential of this novel construct, Hf-BDC was PEGylated and loaded with the PARP inhibitor, talazoparib (T), and the PI3K inhibitor, buparlisib (B) (FIG. 1). The resulting drug-loaded exemplary composition nanomaterial, TB@Hf-BDC-PEG displayed in vitro synergy upon combination with RT and an enhanced ability in both induce DNA damage and inhibit its repair, underscoring its complementary activity.

Translation to an in vivo breast cancer model demonstrated the therapeutic promise of utilizing this nMOF for tumor growth inhibition. This was further supported by immunohistology, where the combination of concurrent RT with TB@Hf-BDC-PEG resulted in increased apoptosis and decreased proliferation compared to all treatment groups, demonstrating its synergistic activity. Additionally, this therapeutic regimen produced no significant side effects, rendering it an alternative candidate to tradition nanomaterials. As such, this work provides the first evidence of nMOFs for enhancing the therapeutic response of DDR inhibitors and RT in cancer therapy.

Synthesis and Characterization of Exemplary Compositions.

From the current library of MOFs, the Hf-containing analog of UiO-66 (Hf-BDC) was chosen as the template material. These Hf-based nMOFs display favorable characteristics for a chemoradiation platform due to properties such as high surface areas for drug loading and secondary building units (SBUs) composed of Hf-oxo clusters, which have previously been demonstrated to effectively absorb X-rays to enhance ROS generation. One particular advantage of the UiO-66 MOF platform is that the overall particle size can be significantly varied through the incorporation of various modulating agents (i.e., acetic acid and benzoic acid), thus allowing these MOFs to be readily synthesized on the nanoscale. This is suggested to result from competitive binding of these ligands with 1,4-dicarboxybenzene (BDC) for coordination to the metal clusters (Zr, Hf) which is thought to decrease the number of nuclei, resulting in larger crystals sizes. In addition to their effect on size, a recent systematic study demonstrated the role of modulators in increasing colloidal stability, therefore preventing high degrees of particle agglomeration with proper optimization.

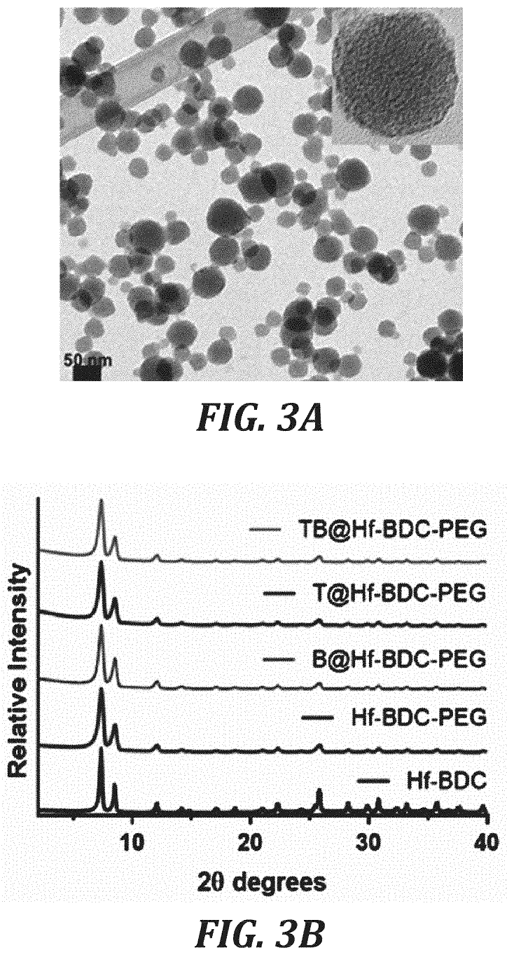

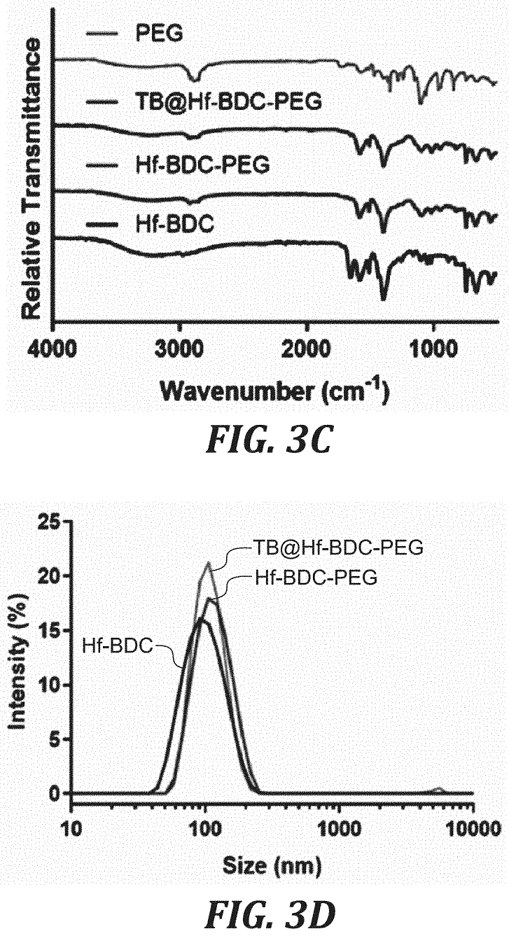

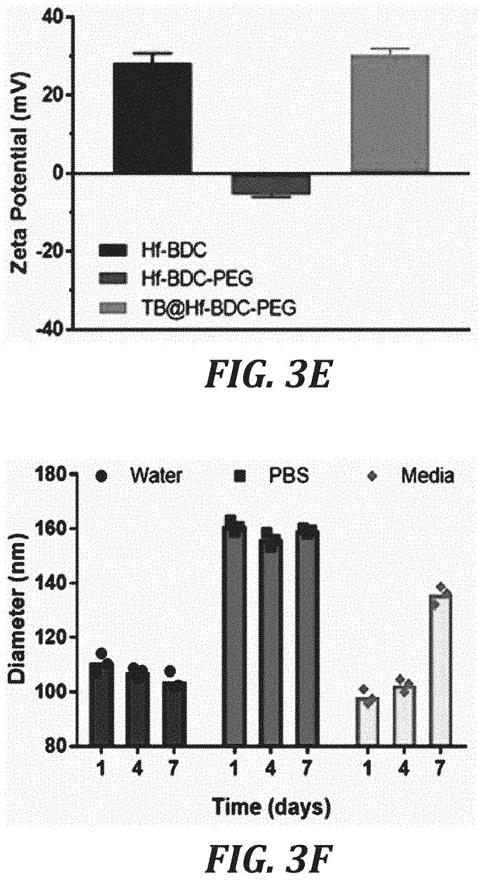

Here, utilizing a modified protocol, nanoparticles of Hf-BDC were quickly synthesized by reaction of hafnium chloride, BDC, acetic acid, and water in DMF at 120° C. for approximately 15 min (Scheme 2). Transmission electron microscopy (TEM) images demonstrate that the as-synthesized nanoparticles have individual sizes ranging from 20-70 nm with an average diameter of 44±14 nm (FIG. 3A). The particle size and size distribution were further validated by dynamic light scattering (DLS), with z-average and polydispersity index values of 92.1±1.63 nm and 0.09±0.01 (FIG. 3D). Fourier transform infrared (FT-IR) spectra displayed peaks associated with Hf-BDC including the strong peak at 1390 cm⁻¹ associated with stretching modes of the carboxylate groups (FIG. 3C). As shown in FIG. 1B, powder X-ray diffraction (PXRD) revealed the expected diffraction pattern associated with the UiO-66 type framework confirming that the Hf-nMOF was successfully produced. Digestion with nitric acid, followed by inductively coupled plasma mass spectroscopy (ICP-MS) analysis revealed that hafnium accounted for 37% of the total weight. To optimize performance for potential in vivo applications, 1,2-distearoyl-sn-glycero-3-phosphorethanolamine-poly (ethylene glycol) (DSPE-PEG) was incorporated as the coating agent for Hf-BDC (Hf-BDC-PEG). This phospholipid-polymer material demonstrates a high degree of biocompatibility, degradability, and an amphiphilic nature allowing it to interact with both the nMOF surface and the surrounding hydrophilic environment. In addition, DSPE-PEG can be readily functionalized with an array of diverse biomolecules allowing for increased specificity and targeting. PEGylation of Hf-BDC with DSPE-PEG resulted in a slight increase in overall size from 92.1 to 109 nm (FIG. 3D) as determined by DLS, where evaluation by TEM indicated no significant changes in particle size compared to bare particles. However, zeta potential analysis revealed a significant reduction in surface charge from 28.4±0.84 to −5.37±0.67 mV, indicating successful PEGylation (FIG. 3E). Further evidence was provided by the appearance of spa C—H, C—C, and C—O stretching bands in the IR spectrum at approximately 2950, 1120, and 1090 cm⁻¹ (FIG. 3C). Furthermore, PXRD analysis following PEGylation revealed the retention of the expected diffraction pattern demonstrating that surface modification does not impact the overall crystallinity of the Hf-BDC nMOFs (FIG. 3B).

Colloidal stability was evaluated for Hf-BDC-PEG under a variety of physiologically relevant conditions. Importantly, Hf-BDC-PEG demonstrated good stability in a variety of physiological media, e.g., water, phosphate buffered saline (PBS), Dulbecco's modified Eagle medium (DMEM). Following incubation in water and PBS, no significant morphological alterations of Hf-BDC-PEG was observed based on DLS measurements (z-averages) (FIG. 3F). However, after 7 days exposure to DMEM, Hf-BDC-PEG undergoes an increase in size from 98 nm to 136 nm. Without wishing to be bound by theory, this was hypothesized to result from the formation of a nanoparticle protein corona overtime, a common occurrence upon nanoparticle exposure to biological fluids, as the uncoated Hf-BDC particles underwent an immediate increase in particle size.

Further analysis by pXRD and TEM reveals a loss of the crystalline nature as the particles are beginning to undergo degradation/corrosion. Despite the ability of PEG to interact with the organic bridging ligand (BDC), particle degradation is likely resulting from removal of the PEG coating on the MOF surface from corrosion overtime. For applications where this is a concern, additional steps should be considered for increasing stability such as the addition of a silica shell or covalent attachment. Here, PEGylation of Hf-BDC serves as a short-term solution for increasing particle stability in various media.

Talazoparib and Buparlisib Loading and Release.

UiO-66 type nMOFs have previously been assessed for their use as drug carriers by loading various cargo including doxorubicin, alendronate, and 5-fluorouracil, among others. However, the majority of these reports were fundamentally focused with only one proceeding to in vivo studies. Here, the drug loading capacity of Hf-BDC-PEG for the two exemplary DDR inhibitors, talazoparib and buparlisib, was evaluated. Encapsulation efficiency (EE %) of talazoparib (Tal or T) and buparlisib (Bup or B) were found to be 77% and 14%, respectively, as determined by high performance liquid chromatography (HPLC). In this exemplary formulation, dual encapsulation of the drugs did not significantly influence their overall drug encapsulation efficiency when compared to individual loading efficiency. Evaluation by TEM and DLS following drug encapsulation demonstrated the TB@HF-BDC-PEG nMOFs retained their overall morphology with no significant increase in size (112±1.49 nm by DLS), whereas zeta potential analysis showed a significant increase in charge (30.4±1.5 mV) (FIG. 3E). Furthermore, PXRD analysis provided evidence that the Hf-BDC nMOFs are retaining their crystalline nature upon the loading process (FIG. 3B).

Regarding MOF drug loading, several studies have observed that drug loading potential is highly governed by the hydrophobicity and hydrophilicity of both the MOF and drug. In this case, the drug with a slightly higher hydrophobicity (Tal log P=2.93, Bup log P=2.13) exhibited higher EE values, corresponding well with the suggested trends. However, it is unknown whether both the drugs are directly loaded into the pores of the Hf-BDC framework or interacting with the organic linkers through π-π interactions of the aromatic portions. Interestingly, these π-π interactions have been previously suggested to contribute to drug loading capabilities in MOFs. Despite the exact loading position of talazoparib and buparlisib being unknown, high T/B loading capacities were achieved.

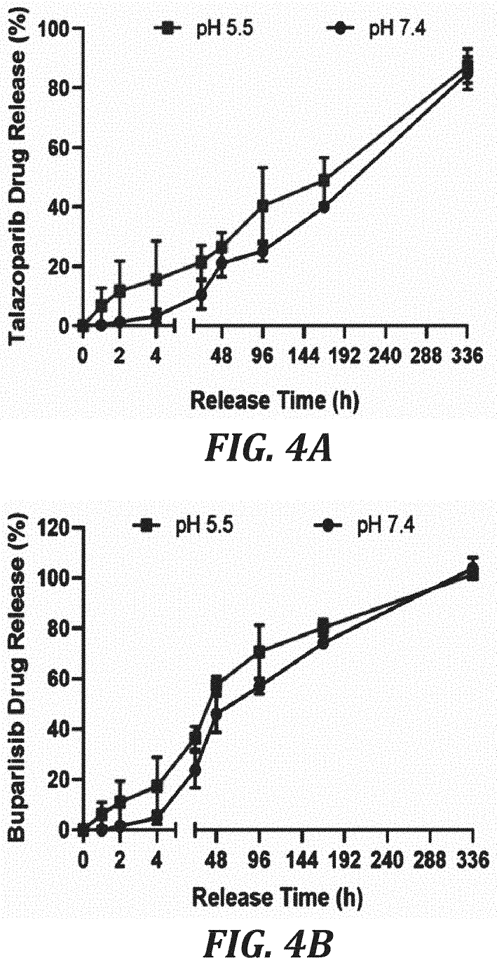

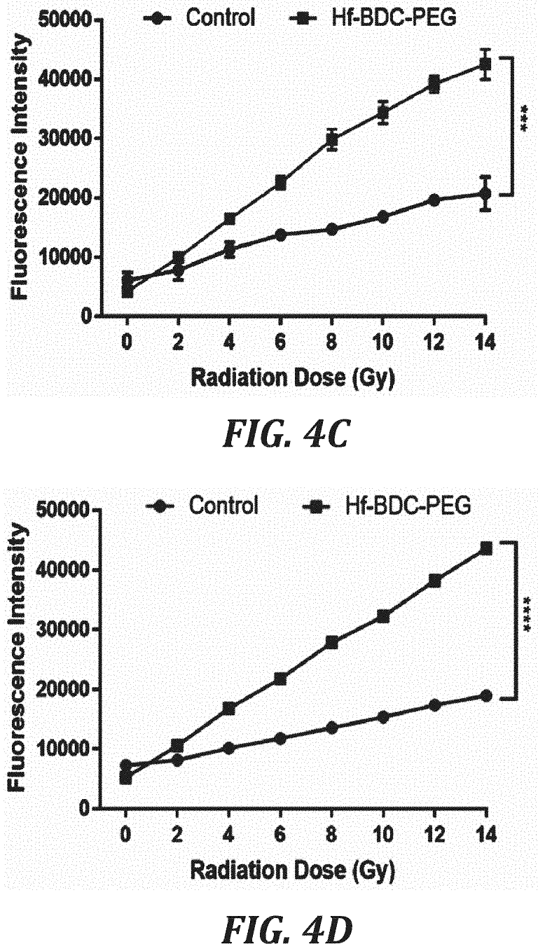

To investigate the influence of pH on drug release, T/B release was tested under simulated physiological pH conditions of 7.4 and 5.5 (PBS, 25° C.). Cumulative drug release curves suggest sustained drug release with no observable burst effect (FIGS. 4A and 4B). Additionally, pH conditions had a slight impact on the release rate of both talazoparib and buparlisib from Hf-BDC nMOFs (FIGS. 4A and 4B). Under pH conditions mimicking circulation (pH 7.5, PBS, 25° C.), approximately 85 and 100% of talazoparib and buparlisib was release after two weeks incubation, with 11 and 24% being released within the first 24 hours (FIGS. 4A and 4B). Comparatively, under more acidic conditions (pH 5.5, PBS, 25° C.), an increased drug release rate is observed with approximately 21% and 37% of talazoparib and buparlisib being released within the first 24 hours, suggesting the exemplary TB@Hf-BDC-PEG nMOFs would have increased sensitivity in acidic tumor environments. These acidic conditions can result in increased positive charges on talazoparib and buparlisib due to protonation, thereby weakening drug-MOF electrostatic interactions and enhancing release. Overall, these results indicate that T/B release from HF-BDC-PEG occurs in a sustained and pH-dependent manner, characteristics which are favorable for in vivo applications.

Hydroxyl Radical and Singlet Oxygen Formation.

The potential of nMOFs to serve as radioenhancers is a relatively new concept. Much of the prior research regarding Hf-based nMOFs for oncology has focused on their utility in photodynamic therapy (PDT), which involves the utilization of non-X-ray (light) source and a photosensitizing (PS) ligand. For these applications, the presence of high atomic number (Z) elements in the SBU is suggested to improve PDT via energy transfer from the high Z-elements to the PS ligand, with subsequent singlet oxygen generation. Recently however, the ability of the electron dense SBUs to directly generate ROS upon exposure to ionizing X-ray irradiation has been discovered. This phenomenon is suggested to occur through the formation of hydroxyl radicals via absorption of X-rays and presents an opportunity to exploit nMOFs for drug delivery and RT enhancement.

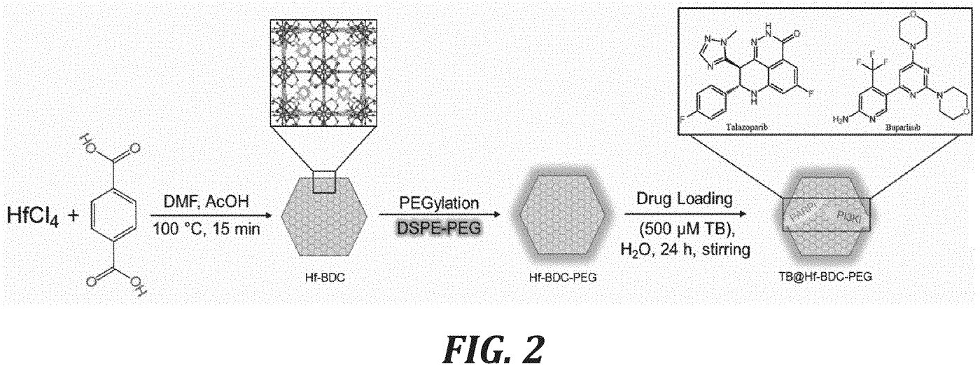

The RT enhancing ability of Hf-BDC-PEG was evaluated by detection of singlet oxygen and hydroxyl radical formation upon X-ray irradiation. Singlet oxygen generation was first assessed using the singlet oxygen sensor green (SOSG) assay. The SOSG reagent is selective for and becomes highly fluorescent upon interaction with singlet oxygen. Thus, the fluorescence intensity increases in response to an increase in singlet oxygen generation. Upon radiation, a linear response in the fluorescence intensity was observed as the radiation dose was continuously increased (FIG. 4C). Hf-BDC-PEG exhibited an increased fluorescent signal overall baseline (water) samples, indicating enhanced generation of singlet oxygen by the nMOF upon irradiation. The ability of Hf-BDC-PEG to increase hydroxyl radical formation upon irradiation was assessed using aminophenyl fluorescein (APF), which becomes highly fluorescent upon reaction with hydroxyl radicals. As expected, a linear increase in fluorescence intensity was again observed as the radiation dose was increased, corresponding to a radiation dose-dependent increase in hydroxyl radical generation (FIG. 2D). Lastly, the ability of Hf-BDC-PEG to interact with X-rays was further supported by the observation of enhanced X-ray attenuation as a function of [Hf-BDC-PEG].

Cellular Uptake and Cytotoxicity.

To evaluate the ability of cells to uptake Hf-BDC-PEG particles, 4T1 murine breast cancer cells were treated with nMOFs encapsulating the hydrophobic, near-infrared fluorescent probe, cyanine 7.5 carboxylic acid (Cy 7.5). Successful loading was confirmed qualitatively as Hf-BDC-PEG particles visually changed color from white to blue upon Cy 7.5 loading (Cy 7.5@Hf-BDC-PEG). This was validated via UV-vis by the appearance of an absorbance peak at 800 nm. Quantification of Cy 7.5 loading via HPLC demonstrated a high encapsulation efficiency of approximately 98%. Qualitative fluorescent imaging demonstrated that Cy 7.5@Hf-BDC-PEG uptake increased over time with a large portion of uptake occurring during the first six hours of incubation. Evaluation of images for maximum fluorescent intensity at each time point suggested a linear increase in uptake until fluorescent signal began quenching after 6 hours, similar to the previous observations. Quantitative analysis of Hf concentration using ICP-MS confirmed that cellular uptake of unloaded Hf-BDC-PEG occurs in a time-dependent manner with a total of 7 ng/10³ cells reached after 24 h.

Taken together, these results indicate the successful uptake of Hf-BDC-PEG by 4T1 cancer cells. After demonstrating cellular uptake, the in vitro toxicity of Hf-BDC-PEG and drug-loaded TB@Hf-BDC-PEG were evaluated in 4T1 cells using the CellTiter-Fluor assay. Cells treated with Hf-BDC-PEG demonstrated no inherent cytotoxicity at concentration ranges between 0-100 ug/mL, with <80% cell viability observed only at 250 μg/mL, suggesting good biocompatibility of the Hf-BDC-PEG nMOF. The low toxicity of Hf-BDC-PEG was further demonstrated by evaluating the biocompatibility on NIH 3T3 (non-cancerous murine fibroblasts) and HepG2 (human liver carcinoma) cells at varying concentrations. As expected, Hf-BDC-PEG had negligible toxicity towards both cell lines with viability dropping below 80% only at 250 μg/mL in the HepG2 cell line. In terms of organ-specific biocompatibility, this observed lack of toxicity is promising, as the liver is the primary organ responsible for the storage and excretion of nanoparticles.

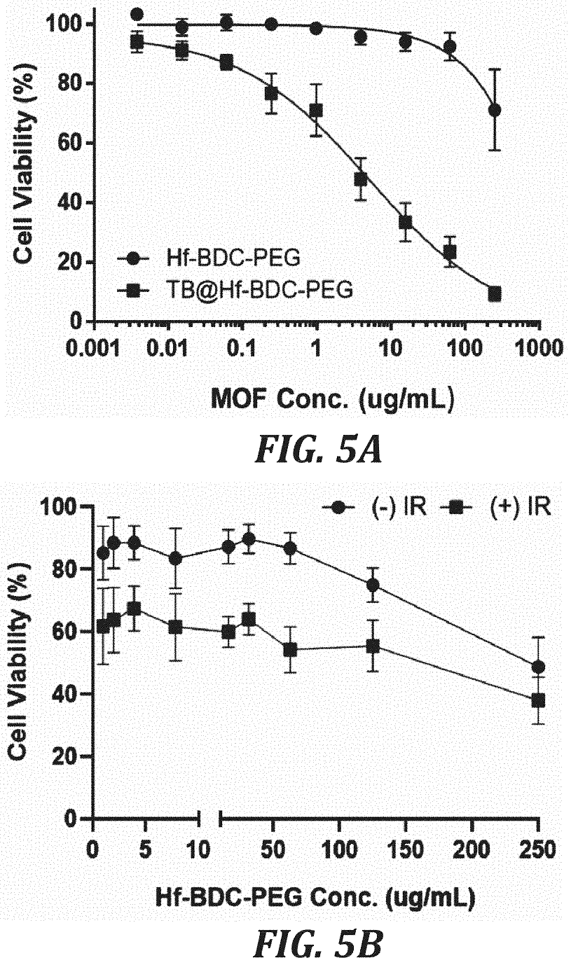

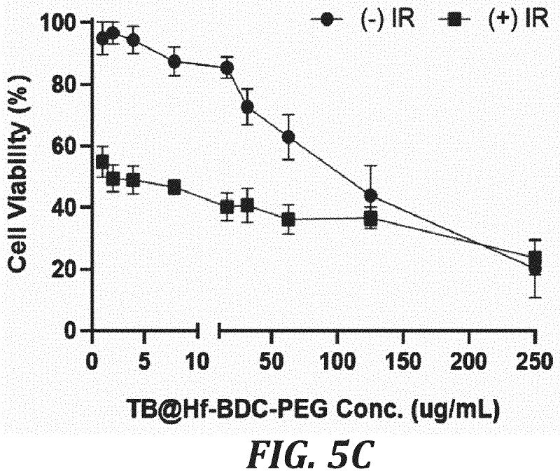

Comparatively, treatment with TB@Hf-BDC-PEG demonstrated obvious cytotoxicity towards 4T1 cells in the tested concentration range (0-250 μg/mL MOF) with a determined nMOF IC₅₀ value of 5.7 μg/mL. This corresponds to loaded talazoparib and buparlisib concentrations of approximately 0.073 μM and 0.007 μM (FIG. 5A). Previously, the inventors reported individual agent IC₅₀ values of 4.7 and 1.4 μM for buparlisib and talazoparib in 4T1 cells, relatively high concentrations where efficacy would require delivery of a large quantity of insoluble drug. Here, direct encapsulation of the DDR inhibitors in the nMOF decreases the drug concentration required for efficacy, indicating synergy between Hf-BDC and the DDR inhibitors. The increase in therapeutic efficacy of TB@Hf-BDC-PEG may be due to the particles providing enhanced transportation and uptake of the drugs into the cells, thereby negating issues related to drug solubility. Furthermore, the observed sustained and controlled release of the drugs ensures continuous drug dosing compared to initial saturation characteristic of free drug administration.

Influence of RT on TB@Hf-BDC-PEG Anticancer Activity.

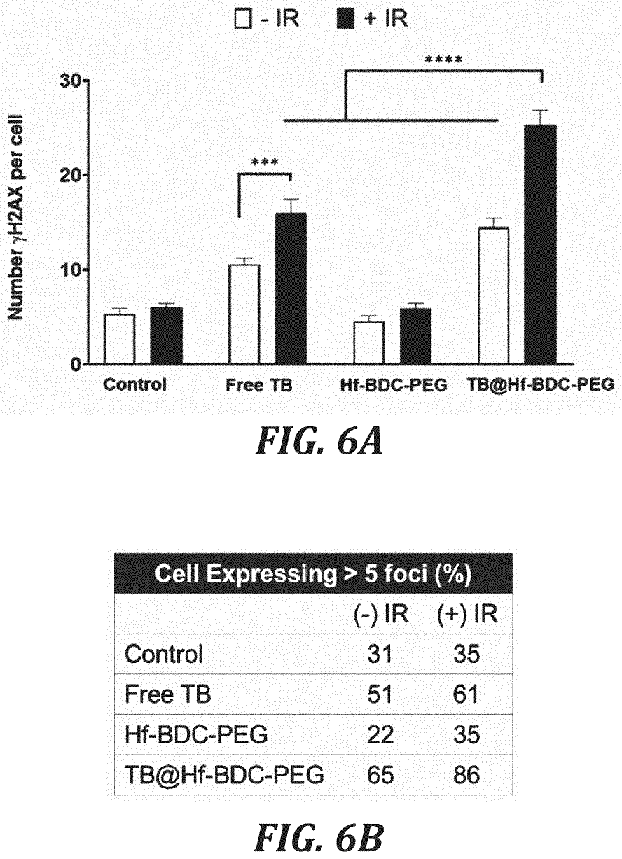

Next, the radiosynergy of the various agents was evaluated in vitro (FIG. 5A). Free TB, Hf-BDC-PEG, and TB@Hf-BDC-PEG were incubated with 4T1 cells at various concentrations for 4 h, followed by irradiation (4 Gy). Seventy-two hours after irradiation, cellular protease activity was evaluated using the CellTiter-Fluor assay as a surrogate for viability. As shown in FIG. 6B, there is some correlation between decreased cell viability and Hf-BDC-PEG concentration. Importantly, combination with irradiation results in a slight decrease in cell viability with enhanced separation occurring particularly at concentrations between 62.5-15.6 μg/mL (FIG. 5B). As the lower concentrations of Hf-BDC-PEG demonstrate minimal toxicity without irradiation, the decreased viability of Hf-BDC-PEG+RT is likely a product of increased ROS generation. Unsurprisingly, cells treated with TB@Hf-BDC-PEG+RT revealed significant therapeutic enhancement compared to no RT treatment (FIG. 5C). Although there is a trend of increasing toxicity with increasing concentration, the synergistic effects of TB@Hf-BDC-PEG become more pronounced and statistically significant at the lower concentrations (FIG. 5C). At higher concentrations of TB@Hf-BDC-PEG, it becomes difficult to assess radiosynergy as both the nMOF and therapeutic drugs have their own intrinsic toxicity, regardless of RT. At the lower concentrations however, minimal toxicity was observed in the absence of RT and significant toxicity (<60%) when RT is combined with the drugs, indicating a high degree of synergistic treatment efficacy. For example, as seen in FIG. 4c, at the lowest dosed TB@Hf-BDC-PEG concentration (0.98 μg/mL MOF, 0.003 μM T, 0.009 μM B) cell survival with and without irradiation was 61 and 95%, respectively. An important aspect of multi-modal therapy is maximizing anticancer effects while minimizing healthy tissue damage. As the lower concentrations of TB@Hf-BDC-PEG elicit limited to no toxicity alone, targeted RT can potentially be utilized to “activate” the synergistic system. As such, this data presents an advantageous option particularly compared to traditional chemotherapeutics for enhancing the therapeutic response.

To further corroborate the radiation and drug synergy induced by the nMOFs, the Chou-Talay method was utilized to determine combination index (CI) values for various fractions affected (fa). These values were then plotted against each other to produce a CI-Fa plot which can be evaluated to determine synergy. From the generated plot, values of CI=1 are additive, those of CI>1 are antagonistic, and those of CI<1 are synergistic. In the absence of irradiation multiple CI values are >1, indicating that the combination of the MOF with the drugs is displaying an antagonistic effect at certain concentrations. Importantly, the addition of irradiation resulted in all CI values being <1, demonstrating synergistic effects at all concentrations.

Influence of TB@Hf-BDC-PEG on DNA Damage and Clonogenicity.

The induction of DNA damage, particularly DSBs, is fundamental to the therapeutic efficacy of radiosensitizing agents. In these studies, RT damages DNA in a specific region at which point DDR inhibitors impart their influence in this specific region, resulting in a biologically driven targeted response. To evaluate the therapeutic efficacy of the Hf-BDC-PEG and exemplary TB@Hf-BDC-PEG, γH2AX was quantified as a surrogate to monitor the extent of DNA DSB induction following radiation. Histone H2AX is a protein that is phosphorylated (γH2AX) in response to DNA damage, which serves as a biomarker for evaluating DSB.

Here, DNA damage was evaluated at 15 min (to visualize maximal damage) and at 24 h (to visualize repair or repair inhibition) after irradiation for samples containing free TB, Hf-BDC-PEG, and TB@Hf-BDC-PEG. Following irradiation, γH2AX foci were identified both as an average number of foci per nucleus and as the percentage of nuclei positive for >5 foci per cell. Fifteen minutes after irradiation, significant increases in DNA damage were observed for all treatment groups in comparison to unirradiated samples. In terms of foci per cell, irradiated free TB and TB@Hf-BDC-PEG induced the largest amount per cell (16 foci/cell). However, this was not statistically different from RT and MOF+RT. This observation is not necessarily surprising as DNA damage has been shown to reach maximum levels 10-30 minutes after irradiation. Interestingly, Hf-BDC-PEG demonstrated the highest increase in the amount of DNA damage occurring over baseline (unirradiated) levels. As ROS is one mechanism by which DNA damage is induced, this provides evidence that the increased ROS generation by the nMOFs is potentially increasing the amount of DNA damage inflicted upon irradiation. Upon reaching maximum levels after irradiation, γH2AX foci disappear as the damage is repaired over the subsequent 24 hours. Previously, reports have correlated lasting DNA damage (24 hours post-irradiation) and clonogenic survival rates (radiosensitivity) with a higher retention of foci (and thus DNA damage) corresponding to lower survival fractions and vice versa. Therefore, γH2AX was evaluated 24 hours after irradiation to quantify the residual levels of DNA damage.

Twenty-four hours after irradiation, treatment with Hf-BDC-PEG revealed relatively few foci remaining (5.9 foci/cell) (FIG. 6B), a significant decreased from the observations after 15 min (14.6 foci/cell). In addition to evaluation of residual γH2AX foci, the percentage of cells expressing γH2AX foci has been correlated to survival rate with cells expressing >3 foci per cell indicative of diminished cell survival. Evaluation of γH2AX foci expression with the radiation control and Hf-BDC-PEG exhibited comparable values, with 36% of cells expressing >5 foci per cell (FIG. 6C). Despite initial increases in DNA DSBs, cancer cell lines with proficient DNA damage repair systems (such as 4T1s) can overcome DNA damage inflicted by Hf-BDC-PEG+RT. Clinically however, The Cancer Genome Atlas (TCGA) project found a high frequency of mutations and alterations to DNA damage repair associated genes and pathways. In these cases, the cell's alternative repair pathways cannot compensate for the DDR defects. Thus, there may be a large population of cancer patients where Hf-BDC-PEG could be effective alone. To test this hypothesis, the inventors evaluated the ability of Hf-BDC-PEG to increase the amount of DNA damage inflicted in a DDR deficient human breast cancer cell line (MDA-MB-436). 24 hours following RT, γH2AX imaging revealed a significant increase in the amount of residual DNA damage, compared to the radiated, untreated control (RT). These findings support the notion that Hf-BDC-PEG can act as a radiosensitizing agent through increased ROS generation. However, to elicit optimal efficacy in DDR proficient cell lines, Hf-BDC-PEG requires supplementary agents to prevent the repair of the increased DNA damage. Importantly, TB@Hf-BDC-PEG resulted in a statistically significant increase in γH2AX foci per cell compared to all treatment groups, inducing the largest amount of damage (25 foci/cell) (FIG. 6B) and exhibiting the largest percentage (86%) of positive cells expressing >5 foci 24 hours after radiation (FIG. 6C). These findings correlate with the known ability of PARP and PI3K inhibitors to enhance radiation sensitivity in cells by suppressing repair of DNA strand breaks. Therefore, through incorporation of DNA repair inhibitors, Hf-BDC-PEG can be used to enhance RT in DDR proficient cancers, as demonstrated by the enhanced efficacy of TB@Hf-BDC-PEG upon irradiation. These results coincide well with the ongoing efforts in personalized medicine, highlighting the optimization of multimodal therapeutics on an individual basis. Here, Hf-BDC-PEG would likely behave synergistically with RT alone in DDR deficient cancers, eliminating further administration of toxic therapeutics. In contrast, patients with DDR proficient cancers would require additional agents to enhanced cell killing and ensure complete tumor control. Taken together, this data supports the utility of combining RT, DDR inhibitors, and Hf-BDC for promoting radiosensitization by directly inducing additional DNA damage and/or inhibiting DNA repair.

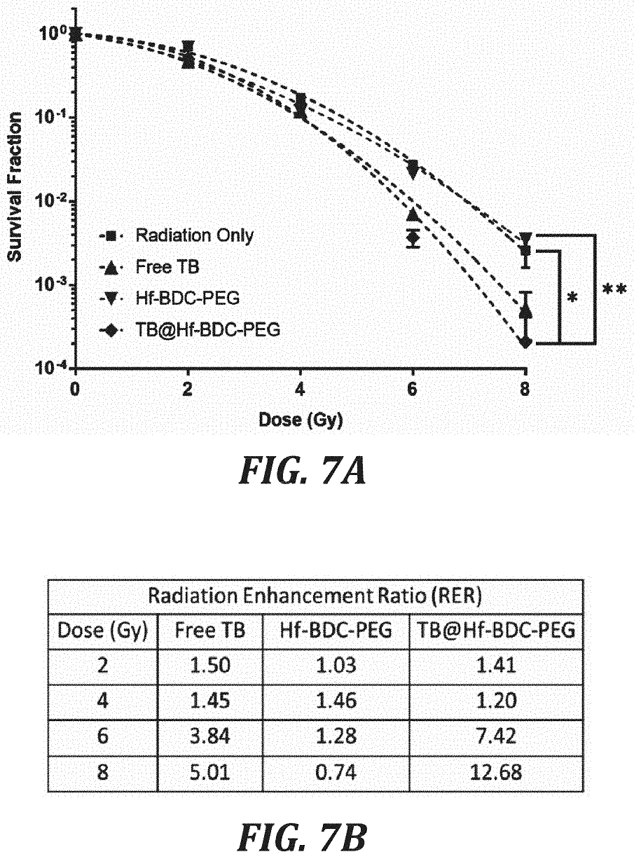

Widely regarded as the gold standard for assessing radiosensitivity, the clonogenic assay evaluates the ability of cells to form clonal colonies after exposure to a treatment. When assessing the efficacy of chemoradiation regimens, this type of evaluation is considered particularly important as cells may not immediately die but may lose their reproductive integrity following treatment. In the context of cancer therapy, the loss of reproduce function nearly equates to non-survival in the same manner as apoptotic cells. As such, evaluation of the same treatment regimen by metabolic assays cannot elucidate reproductive death and often leads to misrepresented cell survival. The in vitro radiosensitization effect of free TB, Hf-BDC-PEG, and TB@Hf-BDC-PEG was evaluated on 4T1 cells treated with increasing doses of radiation. Seven days after treatment, cells were stained and the number of colonies (>50 cells) formed was compared to the number of cells plated in each treatment condition. These experimental results were fitted to the linear quadratic equation and compared in FIG. 7a.

Survival curves suggest that TB@Hf-BDC-PEG creates a more radiosensitive environment over both Hf-BDC-PEG and free TB, demonstrating the synergistic effect and further corroborating the radiosynergy observed via metabolic assays. Comparatively, Hf-BDC-PEG demonstrated minimal enhancement of radiosensitization with the largest separation occurring at the lower radiation doses, aligning well with cell viability and γH2AX results. Additionally, the radiobiological effects of the Hf-BDC nMOFs at a specific dose level was determined by the radiation enhancement ratio (RER), which is defined as the ratio of survival fractions (SF) without and with the nanomaterial at a specific radiation dose.

At the higher radiation doses (6 and 8 Gy), TB@Hf-BDC-PEG outperforms both free TB and Hf-BDC-PEG with RER values of 7.42 and 12.7 respectively.

Despite its widespread use, the clonogenic assay can be cumbersome, time-consuming, and inconvenient for efficient optimization. For this reason, the inventors chose to incorporate a metabolic assay to screen an array of nMOF and TB concentrations with and without radiation. In the development of this platform, and other personalized strategies, there are numerous variables to consider that physically cannot be tested in a practical timeframe utilizing the clonogenic assay. These variables include the optimal concentration of the carrier and drugs required to elicit radiosensitization but not induce toxicity without radiation. Based on this experience herein, the inventors found metabolic assays to first determine a few critical parameters to further investigate followed by the clonogenic assay was an efficient workflow for tailoring these multimodal treatment regimens.

Safety/Toxicity Evaluation of Hf-BDC-PEG nMOF.

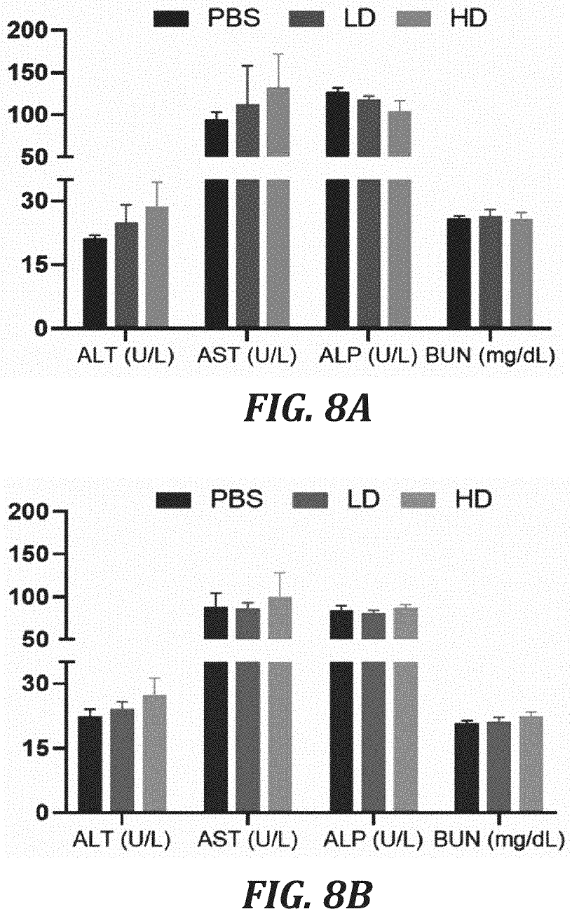

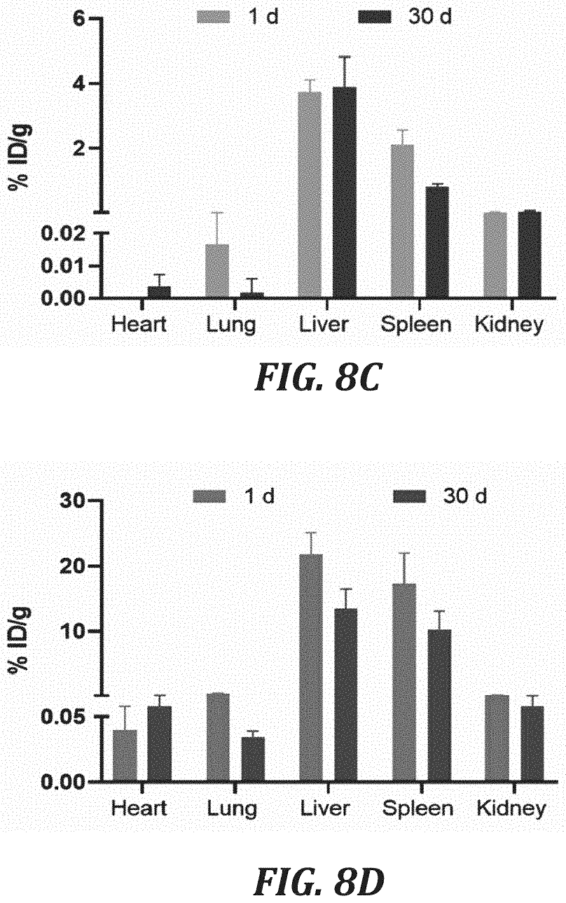

To address the potential clinical translation and feasibility of this platform to function upon intravenous (IV) administration, the hemolytic activity of Hf-BDC-PEG was initially investigated to determine blood compatibility. The assay was performed at varying Hf-BDC-PEG concentrations and up to a maximum concentration of 1000 μg/mL, the hemolysis activity was determined to be <2%, indicating the excellent blood compatibility of Hf-BDC-PEG. However, despite the observed lack of both in vitro cyto- and hemo-toxicity, it is important to recognize that this does not translate to in vivo biocompatibility. To date, very few studies have assessed the in vivo toxicity of nMOFs. Regardless of therapeutic efficacy, the clinical translation of nMOF based materials will remain limited without complete characterization of their biological behavior. Prior to in vivo assessment of therapeutic efficacy, in vivo toxicity was evaluated to assess acute (1 day) and chronic (30 days) toxicity caused by Hf-BDC-PEG after IV administration. Mice were treated with a single dose of either 10 or 55 mg/kg to represent a low and high dose, respectively. Collected blood was sent for clinical chemistry evaluation of alanine aminotransferase (ALT), aspartate aminotransferase (AST), alkaline phosphatase (ALP), and blood urea nitrogen (BUN) (FIGS. 8A and 8B). Administration of Hf-BDC-PEG did not significantly alter markers associated with the liver (ALT, AST, ALP) or kidneys (BUN) at either dose regardless of the timeframe, with all values falling within the range associated with the PBS control, indicating good hepatic and renal tolerance (FIG. 8a, b). Additionally, no noticeable side effects of chronic toxicity were observed based on body weight measurements. Overall, Hf-BDC-PEG demonstrated no obvious acute or chronic toxicity at either concentration rendering it a promising candidate for in vivo translation.

Furthermore, the biodistribution and clearance at 1- and 30-days after intravenous administration was evaluated by quantifying the hafnium concentrations in the heart, lungs, liver, spleen, and kidneys via ICP analysis. Unsurprisingly, one day after intravenous administration of Hf-BDC-PEG, the majority of the particles accumulated in the liver and spleen for both the low and high dose (FIGS. 8C and 8D). Importantly, despite a large portion of particle accumulation in the liver, evaluation of biochemical markers showed no change in activity compared to the control, supporting the notion of good hepatic tolerance. Thirty days following intravenous administration the total amount of accumulated hafnium in all organs decreased in the low and high dose by 19 and 40%, respectively, suggesting clearance of the nMOF (FIGS. 8C and 8D). Evaluation of organ weights revealed no significant variation in the heart, lungs, spleen, or kidneys at 1- and 30-days compared to the control. Interestingly, liver weight increased 1 day after administration, returning to normal levels after 30 days for both the low and high dose of Hf-BDC-PEG. This slight increase in liver weight was previously observed by Horcajada et al and attributed to fast sequestration of nMOFs by clearance organs (liver, spleen) and demonstrated to be fully reversible. In general, the overall biodistribution and rate of organ clearance vary highly and are dictated by the physiochemical properties of MOFs. Certain factors such as ligand lability and lipophilicity, polymer coatings, and inclusion of targeting moieties may directly influence both the distribution of particle accumulation and the clearance rate from individual organs. While the number of parameters evaluated herein is limited, to the best of the inventors' knowledge, the disclosure presents the first report to assess in vivo toxicity and biodistribution of an Hf-BDC nMOF.

Evaluation of In Vivo Tumor Growth Inhibition and Efficacy of TB@Hf-BDC-PEG+X-ray Therapeutic Regimen.

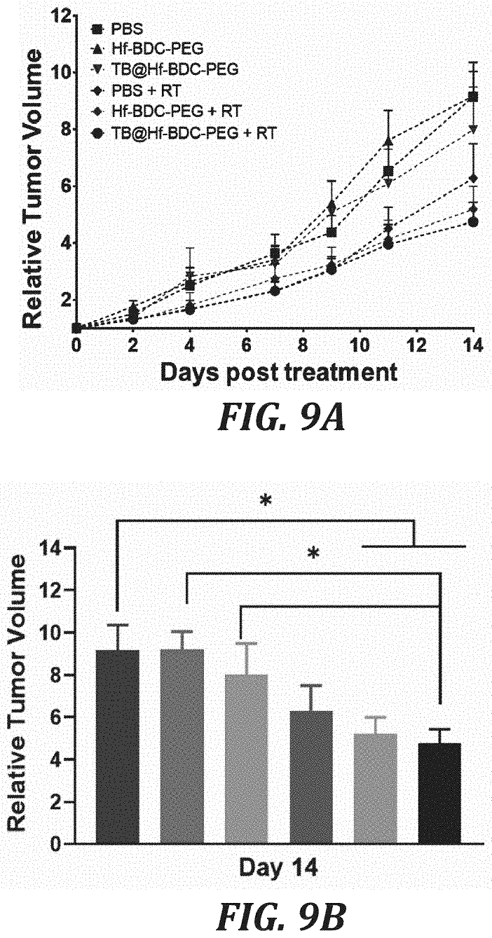

Encouraged by the promising in vitro results and in vivo biocompatibility of Hf-BDC nMOFs, the therapeutic efficacy of both the drug-loaded and non-loaded Hf-BDC nMOF was investigated in the presence or absence of RT on mice bearing 4T1 tumors. A syngeneic mouse model was chosen to assess the response to this chemoradiation regimen in an immunocompetent and more clinically relevant setting. However, these studies were limited due to the rate of tumor growth and degree of tumor burden, as tumor growth occurs at an accelerated rate compared to human xenograft models. Tumor-bearing mice were randomized into six arms (n=5-6 mice) as follows; PBS, Hf-BDC-PEG, TB@Hf-BDC-PEG, PBS+RT, Hf-BDC-PEG+RT, and TB@Hf-BDC-PEG+RT. All mice were injected with the corresponding treatment once daily on days 1-3 followed by three daily fractions of RT (2Gy×3) on days 2-4 for +RT treatment arms. Hf-BDC-PEG and TB@Hf-BDC-PEG were injected IV at a dose of 40 mg/kg Hf-BDC which corresponded to doses of 3 and 0.4 mg/kg for talazoparib and buparlisib, respectively. As shown in FIGS. 9A and 9B, without RT, Hf-BDC-PEG and TB@Hf-BDC-PEG demonstrate negligible antitumor efficacy compared to the control group. PBS+RT resulted in slower tumor growth compared to both Hf-BDC-PEG or TB@Hf-BDC-PEG alone demonstrating the efficacy of radiation. However, by day 9, PBS+RT tumors began displaying a continual increase in size. It is worth noting that for the Hf-BDC-PEG+RT and TB@Hf-BDC-PEG+RT treatment groups, dampening of tumor growth was maintained for a longer duration than PBS+RT. Among all six treatment arms, only Hf-BDC-PEG+RT and TB@Hf-BDC-PEG+RT groups gave significant results compared to the control group (FIG. 9B). Additionally, only TB@Hf-BDC-PEG+RT displayed significantly decreased tumor growth and a significant decrease in tumor weight compared to all the unirradiated groups, further supporting enhanced efficacy. Unfortunately, by day 14 mice had to be euthanized due to significant ulceration in the unirradiated arms, thus hindering the ability to obtain further separation between the PBS+RT and the Hf-BDC-PEG+RT or TB@Hf-BDC-PEG+RT groups. However, based on the observed growth curves, it appears that both nMOF+RT treatment groups would maintain an enhanced degree of tumor growth inhibition at later timepoints.

The use of DDR inhibitors in cancer therapy became a popular approach largely due to the genomic instability characteristic of cancer cells. An estimate ⅓ of TCGA cancer types have defects associated with the various DDR pathways, rendering them especially vulnerable to therapies that exploit the use of DNA damaging agents (i.e., RT and chemotherapies). The DDR pathways are complex, partially overlap, and can adapt to rely on alternative pathways in the event of a loss of function mutation. For example, in cancers that are homologous recombination deficient, such as BRCA 1/2 mutations, cells rely on alternative repair pathways and proteins, such as PARP. The PARP family of proteins are key players in the DNA damage response, detecting strand breaks (SSBs/DSBs) and recruiting the necessary materials for repair. Compared to normal cells, cancer cells have fewer functioning DNA damage repair pathways to rely on and thus single agent PARP therapy can be used to induce synthetic lethality in combination with a BRCA mutation. In these instances, the combination of deficiencies in multiple repair pathways leads to cell death, yet these mutations only occur in a small subset of cancer patients. To expand the utility of PARP inhibitors, therapeutic approaches that mimic HR deficient mutations have been explored. One such approach involves the use of PI3K inhibitors, such as buparlisib, which can decrease BRCA expression and sensitize cells to PARP therapy in BRCA-proficient cells. However, while studies of these drug combinations (and others) have proved promising, a predominant challenge with their clinical utility remains their poor pharmacokinetic properties. Because of this, a PARPi dosing regimen typically involves 1-2× daily administration. To address these limitations various nanocarriers have been investigated, however, nMOFs offer an alternative, superior multi-modal approach that warrants further investigation. In this study, a total of 4 daily treatments (RT and/or MOF) resulted in a significant suppression of tumor growth (by day 14) compared to the control. Taken together, these findings suggest not only that these materials are a promising therapeutic platform, but that they may have the capability of decreasing the required dose, effectively expanding the therapeutic window.

Interestingly, Hf-BDC-PEG+RT demonstrated suppressed tumor growth, a result which was unexpected based on the in vitro results. The failure of in vitro assays such as the clonogenic to effectively predict in vivo responses is predicated on their inability to account for many important variables including immune responses. Taken together with the in vitro assays, this data demonstrates the need to utilize multiple techniques to evaluate the therapeutic response of multimodal agents. Accordingly, in vitro assays should be used as tools for optimizing dosing regimens rather than as a fool proof method to determine clinical applicability. One could predict this response to Hf-BDC-PEG administration when looking at the literature as hafnium oxide nanoparticles have gained recognition for their ability to enhance the dose of RT deposited and generate an immune response upon irradiation. These properties appear to be maintained in MOFs, as researchers found that utilization of a Hf-based nMOF demonstrated significant promise for combined RT and immunotherapy.

In general, the utility of nMOFs for radioenhancement during RT has only recently been explored. In these cases, intratumoral (IT) injection of either Hf-based nanoparticles or nMOFs has resulted in significant tumor growth inhibition with complete regression occurring in some cases. While local administration (IT) is optimal for increasing the therapeutic index, it is often not an appropriate option in many clinical scenarios. In this disclosure, the observation that systemic administration of Hf-BDC-PEG+RT suppressed tumor growth compared to the unirradiated counterpart is a promising result for increasing its versatility and clinical utility. Additionally, the further suppression of tumor growth when utilizing exemplary composition TB@Hf-BDC-PEG+RT showcases the multifunctional utility of a MOF that can both enhance RT and deliver a therapeutic payload.

From the tumor growth trend that was emerging, without wishing to be bound by theory, the inventors hypothesized that diverging intrinsic processes were potentially occurring between the PBS+RT group and the nMOF+RT groups. As such, despite a significant separation between PBS+RT and both nMOFs+RT at day 14, the inventors sought to investigate what was occurring on the biological level. Clinically, tumor regression after therapy is oftentimes undiscernible through physical methods, potentially for months. As in vivo studies typically monitor tumor size by physical methods such as caliper measurements, evaluation of the therapeutic response at early time points will likely prove problematic, disguising the true response. Thus, to further elucidate the influence of the treatments on cancer cells, tumor tissue was extracted and stained for hematoxylin eosin (H&E), cleaved caspase-3 (CC3), and Ki67 (FIG. 9). Staining of tissues sections with CC3 and Ki67, allow for evaluation of apoptosis and proliferation, respectively. As tumors grow, they often develop a necrotic core due to the oxygen and nutrient deprivation which arises from poor blood supply. Both apoptosis and necrosis are forms of cell death, however, apoptosis is considered a sign of programmed cell death which is often tumor-suppressive. In contrast, necrosis has been linked to both tumor progression and aggressiveness. Unsurprisingly, herein it has been observed that tumors from all treatment groups display some degree of a necrotic core, due to the aggressive nature of the 4T1 model.

The extent of apoptosis (CC3 expression) was substantially higher in the nMOF+RT treatment arms in non-necrotic regions. Further evaluation of tissue regions along the tumor periphery reveals little to no apoptosis occurring in the PBS, Hf-BDC-PEG, and PBS+RT treatment arms. Whereas, TB@Hf-BDC-PEG, Hf-BDC-PEG+RT, and TB@Hf-BDC-PEG+RT all exhibited higher CC3 expression in this region, with the latter exhibiting apoptosis throughout the entire tumor. These findings suggest that the aggressive, proliferative nature of this tumor appears to have been stunted in response to treatment. Moreover, while all tumors exhibited some degree of proliferation (Ki67 expression), this was observed to a lesser degree in both nMOF+RT treatment arms, further confirming this hypothesis.

Interestingly, despite the smaller tumor sizes in the PBS+RT treatment group, this was not necessarily reflected in the histology. Here, overall proliferation seemed muted compared to the control and the extent of apoptosis was low. This supports the observation of the PBS+RT tumors displaying continual increases in size by day 9 of the study, suggesting that the initial response to RT was not maintained. An additional consideration is the low energy radiation used in treatment. Aside from potential impact on radiobiologic effectiveness, utilization of low energy radiation results in non-homogenous treatment due to a lack in penetration depth. Taken together, these findings support the combination of TB@Hf-BDC-PEG+RT for synergistically enhancing the therapeutic response of chemoradiation therapy.

Furthermore, as PARP combination therapies are known to exhibit adverse events, some of which can be severe, chronic toxicity was evaluated for all treatment groups. At study end, blood was collected and analyzed for biomarkers associated with hepato-, renal-, and hematological toxicity. Assessment of these biomarkers revealed no differences in hepatic (ALT, AST, ALP) or renal (BUN) toxicity markers between all treatment groups, suggesting high tolerance of the various treatment regimens.

Upon evaluation of complete blood counts for hematological toxicity, only the TB@Hf-BDC-PEG treatment group displayed elevated levels of white blood cells (neutrophils, monocytes, and lymphocytes). Increased levels of these biological components are commonly associated with a systemic immune response. Elevated levels of neutrophils have been proposed to exhibit a pro-cancer effect through promotion of tumor growth, whereas, elevated lymphocyte counts have been correlated to anti-cancer activity.

Interestingly, irradiated groups showed overall blood counts that seem suppressed upon comparison to the unirradiated groups. Additionally, minimal changes in body weight were observed over the course of the study for all treatment groups, further supporting a lack of systemic toxicity. Overall, Hf-BDC-PEG and TB@Hf-BDC-PEG nMOFs in combination with RT, demonstrated no significant renal, liver, or blood toxicity, further supporting its potential clinical utility.

### CONCLUSIONS

In this disclosure, a straight-forward and unique multimodal approach for increasing the therapeutic index of chemoradiation therapy through radiation dose enhancement and delivery of DDR inhibitors was evaluated. Hf-BDC nanoparticles with an average size of 92 nm were synthesized using a rapid modulator-assisted solvothermal method. Surface modification through PEGlyation with DSPE-PEG resulted in nMOFs that displayed high stability (>1 week) under a variety of physiological media. The resulting nMOF were successfully loaded with two hydrophobic DDR inhibitors, talazoparib (77%) and buparlisib (14%) and displayed controlled drug release over the course of 2 weeks. Upon X-ray irradiation, Hf-BDC-PEG efficiently enhanced production of both singlet oxygen and hydroxyl radicals, as detected by the corresponding ROS assays. Functionally, increases in ROS production can lead to increased DNA damage and enhanced radiosensitivity. Here, the increased ROS production corresponded with an observed enhancement in radiosensitivity in vitro visualized through γH2AX with both Hf-BDC-PEG and TB@Hf-BDC-PEG. However, while both nMOFs+RT increased the initial amount of DNA damage, only treatment with TB@Hf-BDC-PEG+RT resulted in sustained inhibition of DNA damage repair, demonstrating the importance of incorporating DDR inhibitors for therapeutic efficacy. Metabolic and colony forming assays confirmed that TB@Hf-BDC-PEG+RT synergistically enhances cytotoxicity and radiosensitization in vitro.

Preliminary toxicity studies displayed a lack of both short- and long-term toxicity after administration of Hf-BDC-PEG concentrations up to 55 mg/kg, providing promise for in vivo translation. Evaluation of biodistribution in the rodent model suggested that the particles can be eliminated over time, with clearance of up to 40% of the accumulated particles occurring after 30 days. Assessment of in vivo efficacy revealed that the TB@Hf-BDC-PEG nMOF results in significant inhibition of tumor growth compared to all unirradiated treatment groups over a 14-day time frame. As RT is a highly effective treatment modality, separation between the RT groups did not become apparent until later time points, an outcome indicative of effective DDR inhibition therapy. Evaluation of immunohistology provided further evidence of therapeutic efficacy where, compared to all groups, TB@Hf-BDC-PEG+RT both increased apoptosis and decreased overall proliferation to a higher extent. Taken together, this in vivo data supports the utilization of TB@Hf-BDC-PEG as a chemoradiation platform.

Multimodal therapies are essential for the successful treatment of cancer and the burgeoning application of DDR inhibitors into these strategies provides a promising approach for exploiting genomic instability. This disclosure provides a novel in vitro and in vivo evidence demonstrating the potential advantages of utilizing nMOFs in combination with DDR inhibitors for augmenting RT. Taken together, these findings demonstrate the utility of nMOFs as a platform to facilitate multimodal approaches for enhancing the oncological therapeutic window.

### B. Radiation-Promoted, Inflammation-Driven Targeting of Chemoradiotherapy Enhancing nMOFs

This example illustrates preparation and characterization of an exemplary nMOF composition comprising a PARP inhibitor and a DNA damaging agent and demonstrates its use in combination with radiotherapy in treatment of cancers.

Colorectal cancer (CRC) is a leading cause of cancer-related death for both men and women, highlighting the need for new treatment strategies. Advanced disease is often treated with a combination of radiation and cytotoxic agents, such as DNA damage repair inhibitors and DNA damaging agents. To optimize the therapeutic window of these multimodal therapies, advanced nanomaterials have been investigated to deliver sensitizing agents or enhance local radiation dose deposition. Herein, the feasibility of employing a radiation-induced inflammation targeting nanoscale metal-organic framework (nMOF) platform to enhance CRC treatment has been demonstrated. These compositions comprise a fucoidan (Fuco) surface coating to preferentially target P-selectin, which is over-expressed or translocated in irradiated tumors. Using this radiation stimulated delivery strategy, a combination PARP inhibitor talazoparib (Tal) and chemotherapeutic temozolomide (TMZ) drug-loaded nMOF was evaluated herein both in vitro and in vivo, as described below. Significantly, these P-selectin targeted nMOFs (TT@Hf-BDC-Fuco) show improved tumoral accumulation over multiple controls and subsequently enhanced therapeutic effects. The integrated radiation and nanoformulation treatment disclosed herein demonstrated improved tumor control (reduced volume, density, and growth rate) and increased survival in a syngeneic CRC mouse model. Overall, as shown herein, the data support the continued investigation of radiation-priming for targeted drug delivery and further consideration of nanomedicine strategies in the clinical management of advanced CRC.

Radiation was used herein to prime tumors for the targeting of a TMZ/Tal-loaded, Fuco-coated Hf-BDC nMOF (TT@Hf-BDC-Fuco) to increase therapeutic efficacy. The CT26.wt cell line was chosen as the CRC model, as the aggressive nature of these cells more closely mimics human disease and the syngeneic nature of the tumor enables use of immunocompetent mice. This formulation resulted in enhanced cellular uptake and tumor accumulation (with and without IR), increased median survival, reduced tumor volume, density, and growth rate, and inhibited the development of a hypoxic/necrotic core. Furthermore, the aggressive treatment regimen produced no toxicity, indicating the potential clinical utility of this combination. Thus, the functionalized, drug-loaded nMOF compositions of the disclosure present an effective therapeutic treatment for cancers, such as CRC.

Cell Culture and Reagents

Murine CT26.wt and bEnd.3 cells were purchased from ATCC and maintained in RPMI or DMEM, respectively, at 37° C. and 5% CO₂. Media was supplemented with 10% (v/v) fetal bovine serum and 1% (v/v) penicillin/streptomycin. Further cell authentication and Mycoplasma testing has not been performed. Cells were used within 20 passages. Talazoparib and temozolomide were purchased from MedChemExpress (Monmouth Junction, N.J., USA). HPLC grade acetonitrile and methanol were purchased from Fisher Scientific (Hampton, N.H., USA). Cyanine7.5 carboxylic acid was purchased from Lumiprobe (Hunt Valley, Md., USA). Phosphate buffered saline (PBS) 1X was purchased from Corning Inc. (Corning, N.Y., USA). DAPI (4′,6-Diamidino-2-Phenylindole, Dilactate), Alexa Fluor™ 488 Phalloidin, RPMI 1640, DMEM (high glucose, pyruvate) and Geltrex (LDEV-Free Reduced Growth Factor Basement Membrane Matrix) were purchased from Thermo Fisher Scientific (Carlsbad, Calif., USA). Fucoidan (Fucus vesiculosus >95%) and dextran sulfate sodium salt (MW 7,000-20,000) were purchased from Sigma Aldrich (St. Louis, Mo., USA). Glacial acetic acid, terephthalic acid (1,4-benzenedicarboxylic acid—BDC), DMF, hafnium (IV) chloride, and recombinant TNF alpha were purchased from Fischer Scientific.

Surface Modification and Drug Loading of Hf-BDC nMOF

nMOFs were suspended in either 18 MΩ water (generation 1) or methanol (generation 2) at 5 mg/mL depending on colloidal stability. nMOF redispersed in water at 5 mg/mL was combined with fucoidan or dextran (both at 1 mg/mL in 18 MΩ water) at a 1:1 v/v ratio, sonicated for 1 hour, and filtered. nMOF redispersed in methanol at 5 mg/mL was combined with fucoidan or dextran (both at 1 mg/mL in 18 MΩ water) at a 1:1 v/v ratio, sonicated for 15 mins and stirred open overnight. After evaporation nMOFs were redispersed to 2.5 mg/mL nMOF in water and subsequently filtered. For drug loading, all steps remained the same except that drug dissolved in DMSO (Tal and/or TMZ or Cyanine 7.5) was added to the nMOF in water or methanol, sonicated for one hour, and washed by centrifuge twice (collecting the supernatant for quantification) before continuing with either surface coating procedure.

P-Sel Expression

P-sel expression was evaluated in vitro using bEnd.3 and CT26.wt cells. 5×10⁴ bEnd.3 cells were plated into 4-well chamber slides, allowed to settle overnight, irradiated with 6 Gy, fixed 20 hrs later, and stained for P-sel (1/50 dilution) and DAPI. The same protocol was repeated in CT26.wt cells (1.5×10⁵ cells/well). P-sel expression was also evaluated in CT26.wt tumors harvested from BALB/c mice. Subcutaneous CT26.wt tumor xenografts were established by mixing an equal volume of cell suspension (2×10⁶ cells/mL) with Geltrex and injecting 50 μL (5×10⁴ cells) into both hind flanks of BALB/c mice (Charles River Laboratories, Wilmington, Mass.). Twenty days later, mice (n=3) were irradiated with 0, 2, or 6 Gy and tissue harvested at 4- or 24-hrs post-irradiation (IR). Tissues were prepared for histologic examination as described in the histology section.

In Vitro Cytotoxicity

Free drug cytotoxicity was investigated with Tal and TMZ as individual and combination agents. To determine the optimal TMZ:Tal ratio, CT26.wt cells were plated at 4×10³ cells per well in a 96-well plate and allowed to settle overnight. Cells were then treated with TMZ (0, 10, 30, 150, 300, 600 or 1000 μM), Tal (0, 1, 5, 10, or 100 μM), or TMZ:Tal at each of the concentrations combined (ratios ranging from 1000:1 to 1:10) and incubated for 72 hrs. Combination indexes (CIs) were determined for all combinations (including only those points on the curve with Fa values between 0.1 and 0.9) using CompuSyn (ComboSyn Inc., Paramus, N.J., USA).

Clonogenic Assay

One million CT26.wt cells were seeded into six T25 flasks and allowed to settle overnight. 24 hrs later, flasks were treated as follows: no treatment, Hf-BDC-Fuco, Tal@Hf-BDC-Fuco, TMZ@Hf-BDC-Fuco, TT@Hf-BDC-Fuco, or Free Tal/TMZ. The TT@Hf-BDC-Fuco nMOF was diluted 200-fold from the stock (2.5 mg/mL Hf-BDC-Fuco, 7.07 mM TMZ, and 272 μM Tal). Individually loaded nMOFs and free Tal/TMZ were treated to match their counterpart in the TT@Hf-BDC-Fuco treatment arm. After incubating for 20 hrs, cells were washed once with PBS, trypsinized, seeded into 6-well plates, and irradiated (CellRad X-Ray Cabinet Irradiator, Precision X-Ray Irradiation, 130 kV, 5 mA, 0.5 mm aluminum filter, ˜1.2 Gy/min). The number of cells seeded varied based on the dose of radiation (0, 1, 2, 3, 4, 5 Gy) and drug treatment conditions ranging from 100 cells/well to 1500 cells/well. Seven days later, colonies were washed with PBS, fixed with 4% v/v formalin, and stained with 0.01 mg/mL crystal violet dye. After staining, plates were washed with DI water, dried, and colonies counted. First, the plating efficiency (PE) of each treatment was calculated by dividing the number of colonies formed by the number of cells seeded. Next, the PE of each treatment was divided by the PE of the unirradiated counterpart to determine the survival fraction (SF).

Xenograft Tumor Inoculation and Growth

All animal studies were approved by and conducted in accordance with the guidelines of the Institutional Animal Care and Use Committee (IACUC) of Oregon Health and Sciences University. Subcutaneous CT26.wt tumor xenografts were established by mixing an equal volume of cell suspension (2×10⁶ cells/mL) with Geltrex and injecting 50 μL (5×10⁴ cells) into the right flank of BALB/c mice (Charles River Laboratories, Wilmington, Mass.). For all tumor studies, body weight and average tumor volume (½×length×width²) measurements were taken at least three times weekly. When endpoints were reached per IACUC guidelines [(1) tumor ulceration, (2) weight loss reached, (3) 20% of starting weight, or (4) any diameter reached 2 cm] mice were euthanized by cervical dislocation under anesthesia. Relative tumor volume curves were developed to compare starting tumor volume to the volume measured at each day. Kaplan-Meier curves were developed for each treatment group to examine differences in survival. For all imaging studies and when noted, mice were anesthetized using 2-3% isoflurane (Piramal Enterprises Limited, Telangana, India).

Efficacy and Survival Study

Eleven days after inoculation, 28 mice were randomized evenly (n=7) into four treatment groups with average tumor sizes of 55-68 mm³. Treatment groups were as follows: (1) Hf-BDC-Fuco, (2) Hf-BDC-Fuco+RT, (3) TT@Hf-BDC-Fuco, or (4) TT@Hf-BDC-Fuco+RT. Particles were administered via tail vein injection b.i.d. five consecutive days starting on day zero. nMOF and fucoidan concentrations were 9.25 and 1.85 mg/kg per administration and the corresponding TMZ and Tal concentrations were 4.4 and 0.36 mg/kg. Mice in groups two and four were irradiated with 2 Gy on days zero and two for a total of 4 Gy. Radiation was delivered (CellRad, Precision X-Ray Irradiation, 130 kV, 5 mA, 0.5 mm aluminum filter, ˜1.2 Gy/min) selectively to tumors using half-moon cutout lead shields (Precision X-Ray, North Branford, Conn.). Terminal blood samples were collected via cardiac puncture for clinical chemistry, performed by IDEXX laboratories. Organs were collected, weighed, and fixed. Formalin-fixed tumors obtained from days 14 (all treatments) and 21 (all treatments except control) were submitted to the OHSU Histopathology Shared Resource for procedures described in Tissue Histology. Tumor mass was divided by caliper tumor volume to obtain tumor density. For mice was sacrificed due to ulceration, tumors were not included in average tumor density calculations. Tumor volumes were converted to a base-10 logarithmic scale and linear fits were applied to each treatment group. The resulting slope represents the average tumor growth rate for each group. Tumor growth inhibition percent was determined using the tumor growth rate values applied to the formula (1−(growth rate of treatment group)/(growth rate of control group))×100%.

Tissue Histology and Stain Quantification

Tissue was formalin (10% v/v) fixed for 24 hrs and then transferred to 70% ethanol. Samples were then processed and stained by by the OHSU Histopathology Shared Resource Core; stains included P-sel (RB40.34, BD Biosciences, 0.2 mg/mL at 1:100 dilution), CD31 (ab28364, Abcam, supplier recommendations), and Hematoxylin and Eosin (H&E). Imaging was performed on a Zeiss Axio Scan.Z1 Slide Scanner by the OHSU Advanced Light Microscopy Core at 20×(P-sel & CD31) or 40×(H&E). Whole-tumor images were analyzed in FIJI using color threshold. For any given marker (H, E, P-sel, CD31) the RGB settings utilized to select the entire tissue and the marker-stained tissue were applied uniformly to all images. After applying the threshold settings, tissue was selected and area measured. For each image the area stained was divided by the whole tissue area to determine a fraction of tissue stained for each marker.

Statistical Analysis

All data are expressed as mean±SEM. Statistical differences and significance were evaluated using two-way ANOVA with multiple comparisons, one-way ANOVA with multiple comparisons, or logrank (Mantel-Cox) test for survival curves in the GraphPad Prism 8. p<0.05 was considered statistically significant and represented by *.

Synergistic Combination of Talazoparib and Temozolomide

Viability experiments were performed to assess and identify synergistic ratios of TMZ and Tal in CT26.wt cells. Strong synergy (CI values less than 0.35) was observed across high, medium, and low total drug concentrations (31, 155, 310 μM) at a 30:1 TMZ:Tal ratio indicating a robust synergistic effect for this combination.

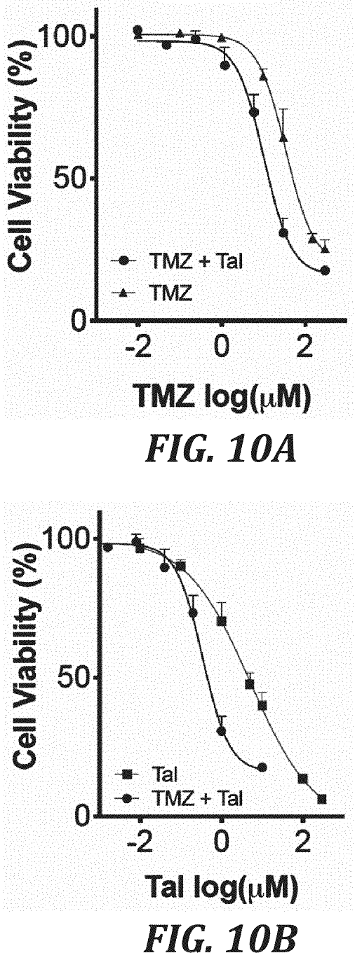

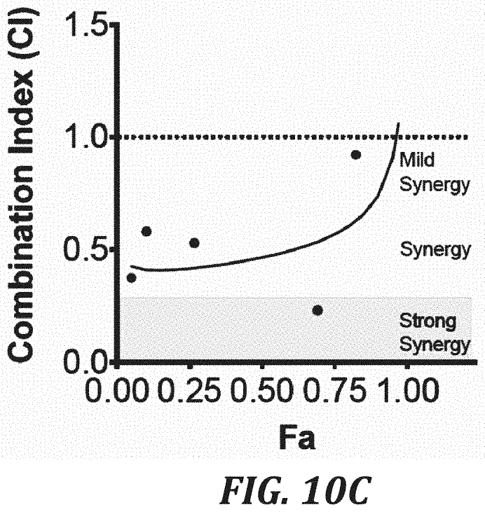

Once a ratio (30:1, TMZ:Tal) was selected, further cytotoxicity and synergy evaluation was performed. Viability curves and subsequent IC₅₀ values were determined for Tal, TMZ, and TMZ:Tal at a 30:1 ratio (FIGS. 10A-C). Tal was found to be moderately effective as a single agent (IC₅₀=5.1 μM) while TMZ was less effective (IC₅₀=34 μM). Importantly, combining TMZ and Tal produces an overall IC₅₀ value of 10.7 μM, correlating to concentrations of 0.35 μM Tal and 10.35 μM TMZ. Compared to their individual drug counterparts, these were IC₅₀ decreases of 14.6- and 3.3-fold for Tal and TMZ respectively, demonstrating increased efficacy of the combination. Synergy was found at all tested concentrations using the Chou-Talalay method (FIG. 10C). Despite the oral bioavailability of Tal and TMZ, solubility limitations of Tal make it an ideal candidate for nanoparticle encapsulation.

Characterization of TT@Hf-BDC-Fuco

To enhance solubility and circulation, the drugs were encapsulated in a nMOF (Hf-BDC). This nMOF was coated with fucoidan (Hf-BDC-Fuco) as an inflammation-targeting (P-sel) agent or dextran (Hf-BDC-Dex), a non-sulfonated polysaccharide as a non-targeting control.

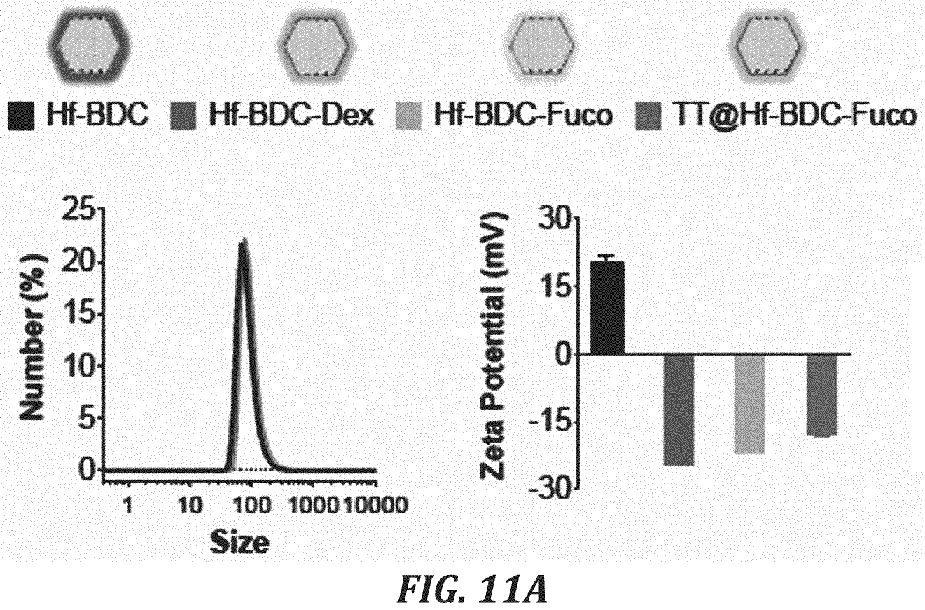

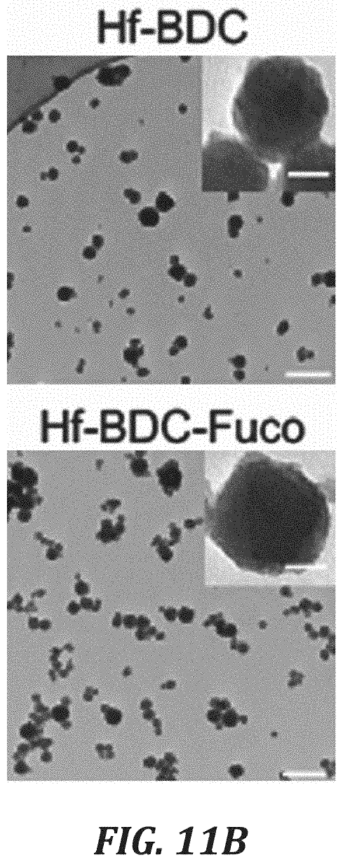

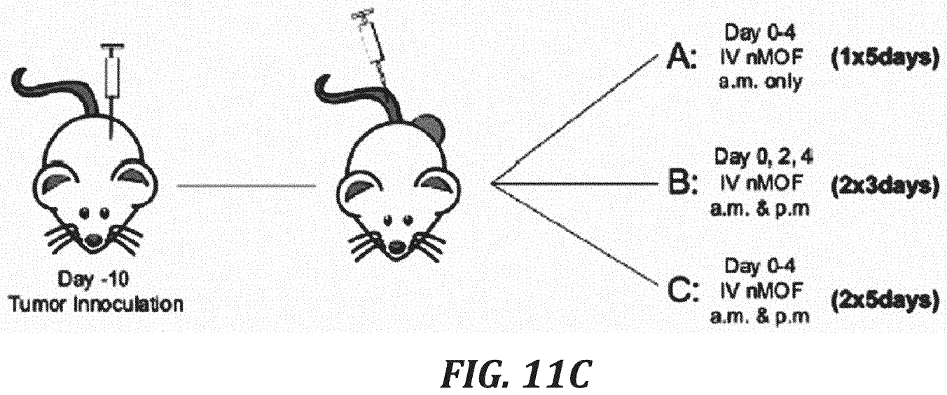

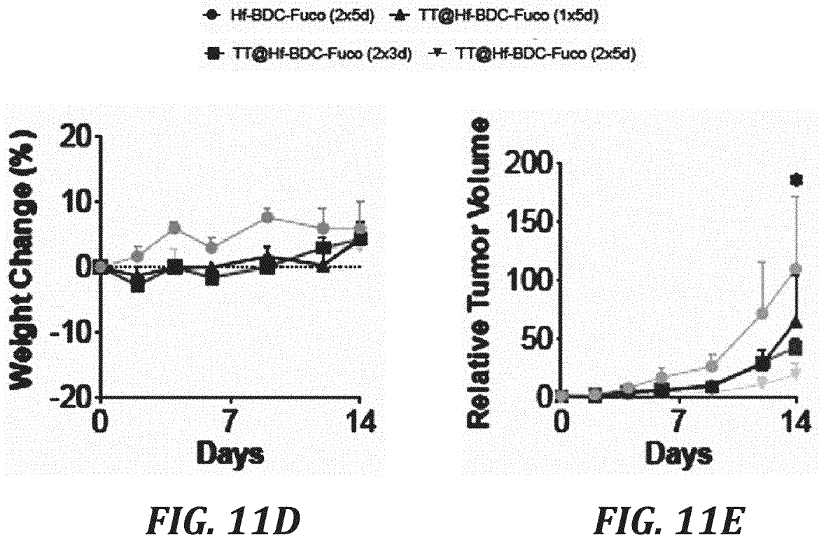

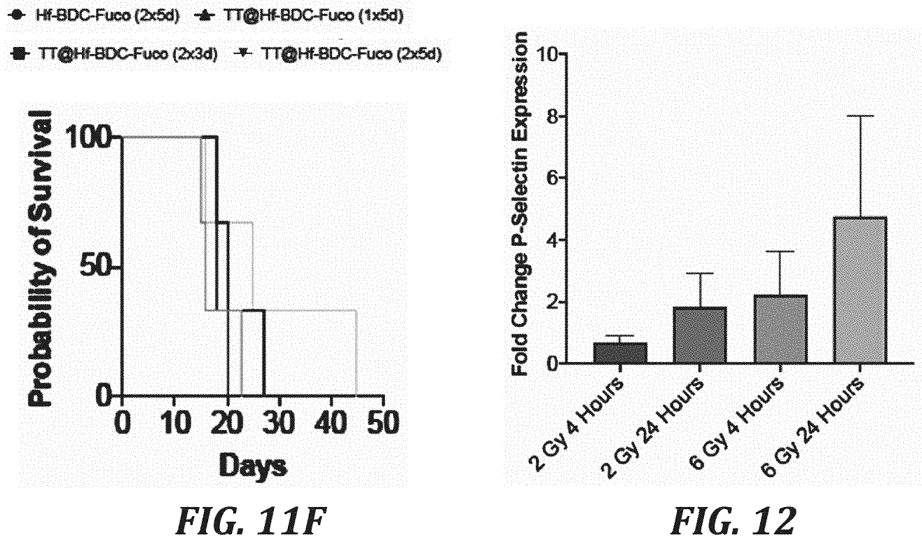

Evaluation of the various Hf-BDC nMOFs by dynamic light scattering (DLS) showed the size of an uncoated nMOF to be ˜84 nm, with each coating and drug loading slightly increasing the nMOF size (FIG. 2A; Supplementary FIG. 3 and Table 2). Both Hf-BDC and Hf-BDC-Fuco revealed size ranges of 30-70 nm by transmission electron microscopy (TEM) (FIG. 11B). Bare Hf-BDC nMOFs demonstrated a positive potential and all polysaccharide-coated nMOFs (Hf-BDC-Dex, Hf-BDC-Fuco, TT@Hf-BDC-Fuco) possessed negative potentials (FIG. 11). Notably, this data agrees with previous iterations of this platform. Furthermore, minimal differences were observed between the two coating strategies. Both drugs loaded effectively in the nMOF with average encapsulation efficiencies (EE) of 90% and 54% for TMZ and Tal.

To confirm the efficacy and determine the optimal in vivo treatment regimen of the TT@Hf-BDC-Fuco nMOF, a proof-of-concept study was performed. Tumor-bearing mice were administered TT@Hf-BDC-Fuco (1 dose×5 days (d), 2×3 d, or 2×5 d) or Hf-BDC-Fuco (2×5 d) and evaluated for weight changes, tumor growth inhibition, and survival (FIG. 2C). All regimens were non-toxic, with the two most aggressive regimens (2×3 d and 2×5 d) demonstrating a significant reduction in relative tumor volume compared to the control nMOF (FIGS. 11D-F). Furthermore, median survival was increased from 16 to 25 days with the 2×5 d treatment regimen. As such, this treatment regimen was selected for further investigation with radiation.

In vitro and in vivo P-sel Expression and Targeted Uptake To leverage the P-sel targeting moiety Fuco, baseline and IR-stimulated P-sel expression was investigated in cancer (CT26.wt) and endothelial (bEnd.3) cells. Prior to IR, cancer cells displayed strong puncta in the cytoplasm with minimal diffuse cytoplasmic staining (FIG. 12). After 6 Gy, puncta are less identifiable and a more diffuse cytoplasmic stain is apparent, indicating translocation. Endothelial cells demonstrate a similar trend of P-sel translocation after 2 Gy.

The ability of radiation to induce an inflammatory environment and translocate P-sel expression in vitro was confirmed in vivo by evaluating the changes in P-sel and CD31 (endothelial cell marker) in irradiated tumors. Mice were exposed to one of three radiation doses (0, 2, 6 Gy) and tissue harvested either 4 or 24 hrs after radiation. With the exception of 2 Gy—4 hrs, both increasing radiation dose (2 to 6 Gy) and time post-IR (4 to 24 hrs) resulted in relative increase in fractional P-sel expression. Fractional P-sel and CD31 expression was determined by dividing the area of tissue stained by the total area of tissue from whole tumor slices. Qualitative differences for both markers could also be observed at higher magnification. In analysis of a tumor from the 6 Gy—24 hrs group, increases were noted for both P-sel (7.07-fold) and CD31 (2.05-fold) in sequential slices. Contrastingly, 2 Gy—4 hrs demonstrated a reduction in P-sel (0.66-fold) and slight increase (1.18-fold) in CD31 while 2 Gy—24 hrs and 6 Gy—4 hrs demonstrated increases in P-sel (3.82- and 4-fold) and slight decreases in CD31 (0.78- and 0.91-fold).

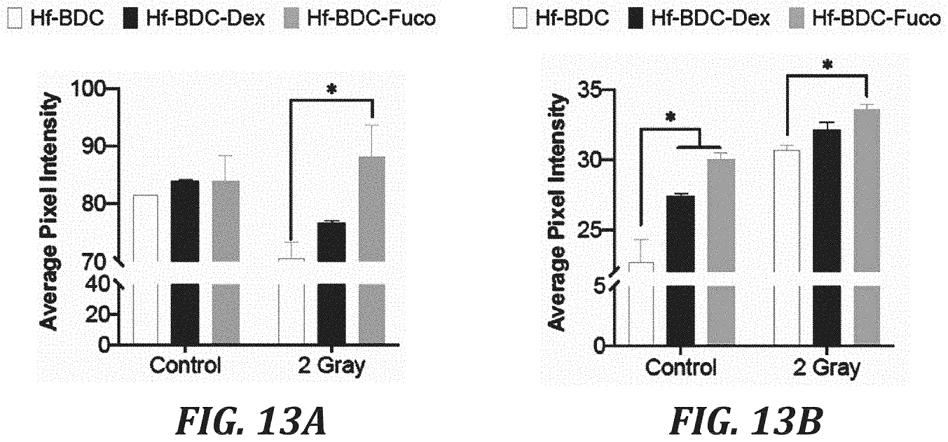

Utilizing a low dose of IR (2 Gy), the ability of −Fuco to enhance nanoparticle homing and uptake was evaluated. Six hours after treatment, cells (CT26.wt or bEnd.3) were incubated with media, Cy7.5@Hf-BDC-Fuco, Cy7.5@Hf-BDC-Dex, or Cy7.5@Hf-BDC overnight. Employing average pixel intensity as a surrogate for particle uptake, surface modification alone is observed to increase uptake in endothelial cells, but not CRC cells (FIGS. 13A and 13B). However, treatment with 2 Gy significantly increases uptake of Cy7.5@Hf-BDC-Fuco compared to the Cy7.5@Hf-BDC in both cell lines. For irradiated CRC cells, but not irradiated endothelial cells, uptake was reduced for both Cy7.5@Hf-BDC and Cy7.5@Hf-BDC-Dex formulations compared to baseline (Cy7.5@Hf-BDC without IR).

In Vitro Toxicity

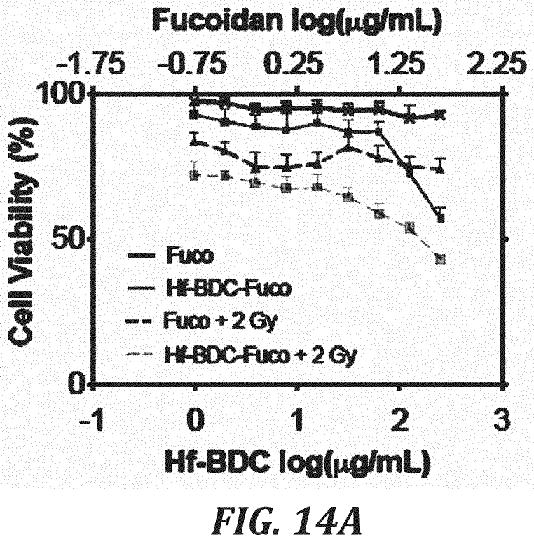

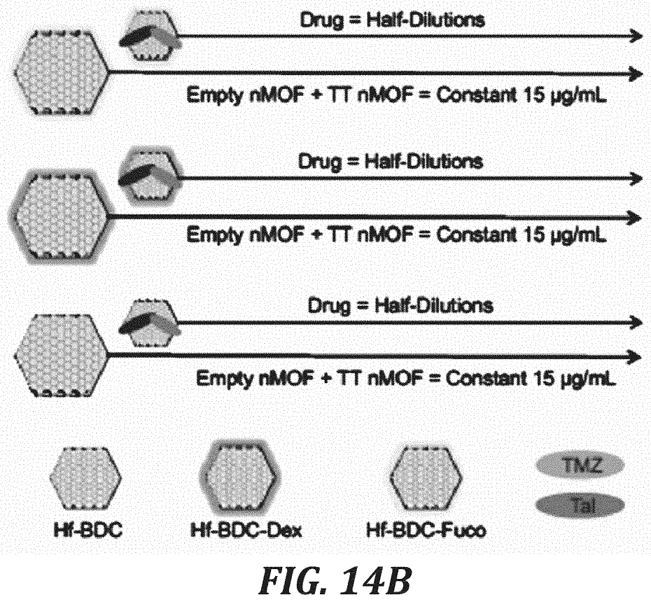

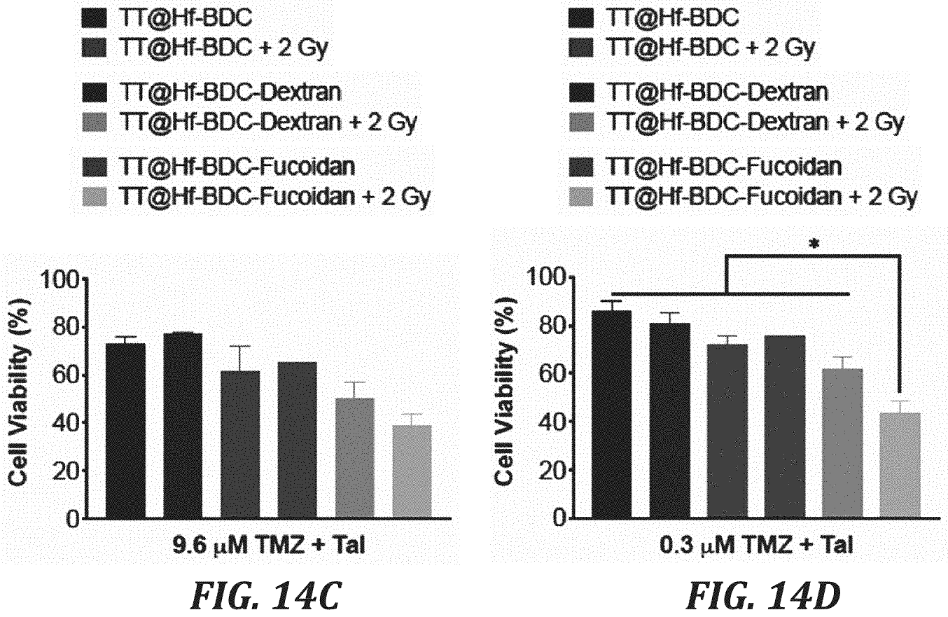

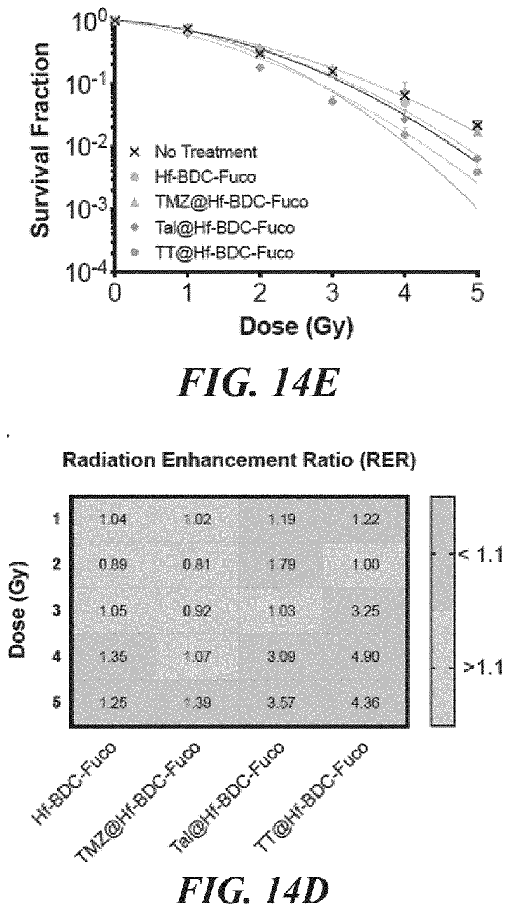

CT26.wt cells were incubated with free Fuco or Hf-BDC-Fuco and treated with 0 or 2 Gy to determine the influence of Fuco on the cellular response to IR. Free Fuco concentrations were chosen based on the amount of Fuco coating in each nMOF treatment. Following 2 Gy, cell viability decreased approximately 20% across all Fuco concentrations tested (FIG. 14A). Importantly, Fuco coating does not inhibit radioenhancing ability of Hf-BDC at any of the concentrations tested. A nMOF concentration of 15 μg/mL was chosen for subsequent studies, for its lack of baseline toxicity and radioenhancing potential.

Next, CT26.wt cells were treated with TT@Hf-BDC, TT@Hf-BDC-Dex, or TT@Hf-BDC-Fuco and radiation to further examine the influence of surface coating. For these studies, nMOF concentration was kept constant (15 μg/mL Hf-BDC) while total drug concentrations ranged from 9.6-0.075 μM at a 1:1 ratio (FIG. 14B). Among unirradiated conditions, cells treated with TT@Hf-BDC-Fuco (solid blue line) demonstrate the greatest cytotoxicity compared to the other −IR treatments (black and purple solid lines). Interestingly, TT@Hf-BDC-Fuco −IR was equally efficacious to irradiated TT@Hf-BDC and TT@Hf-BDC-Dex treated cells. Furthermore, TT@Hf-BDC-Fuco+IR resulted in the strongest cytotoxicity compared to all other treatment groups, suggesting P-sel targeting is enhancing efficacy. Closer evaluation of a single high and low total drug concentration (9.6 and 0.3 μM) revealed the potential for highly cytotoxic doses in non-targeted nMOFs to overcome increased delivery by targeted particles. The lower concentrations led to significant decreases in viability of the TT@Hf-BDC-Fuco+IR treatment group versus all other treatments whereas the higher concentration did not (FIGS. 14C and 14D). Although obtained independently, comparison of Hf-BDC-Fuco and TT@Hf-BDC-Fuco studies demonstrate the cytotoxic influence of drug-loading both with and without IR.

As metabolic-based viability assays do not evaluate radiobiologic cell death, additional screening was performed using the clonogenic assay. Cells were exposed to various treatments (control, Hf-BDC-Fuco, TMZ@Hf-BDC-Fuco, Tal@Hf-BDC-Fuco, or TT@Hf-BDC-Fuco) and increasing doses of radiation (0, 1, 2, 3, 4, 5 Gy). As seen in FIGS. 5E and F, as radiation dose increases, TT@Hf-BDC-Fuco displays enhanced radiosensitization compared to other treatments. Further evaluation of individual radiation doses reveals that compared to the control, TT@Hf-BDC-Fuco significantly decreases survival at 3 and 5 Gy.

Biodistribution

Following in vitro studies, baseline and radiation-enhanced Hf-BDC accumulation was investigated. Four hours after receiving 6 Gy (or no IR), tumor-bearing mice were administered Cy7.5@Hf-BDC-Fuco which were allowed to accumulate for 20 hrs followed by ex vivo fluorescence imaging. Results revealed a slight increase in tumoral Cy7.5@Hf-BDC-Fuco accumulation after IR, indicating potential targeting. Based on the enhanced in vitro accumulation of Hf-BDC-Fuco without radiation, a follow-up study assessed the enhanced accumulation against an untargeted negative control (Hf-BDC-Dex). Here, 24 hrs following 6 Gy, tumor-bearing mice were administered Cy7.5@Hf-BDC-Fuco or Cy7.5@Hf-BDC-Dex which were allowed to accumulation for an additional 24 hrs. In this case, Cy7.5@Hf-BDC-Fuco displays higher tumoral accumulation than Cy7.5@Hf-BDC-Dex both with and without IR.

In Vivo Efficacy and Survival

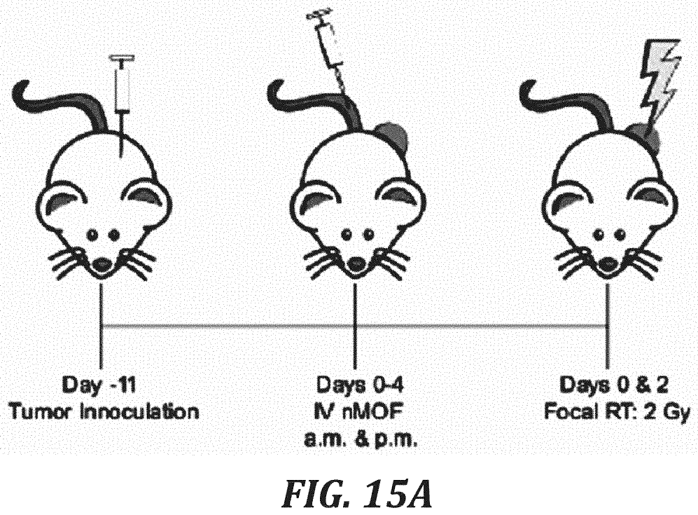

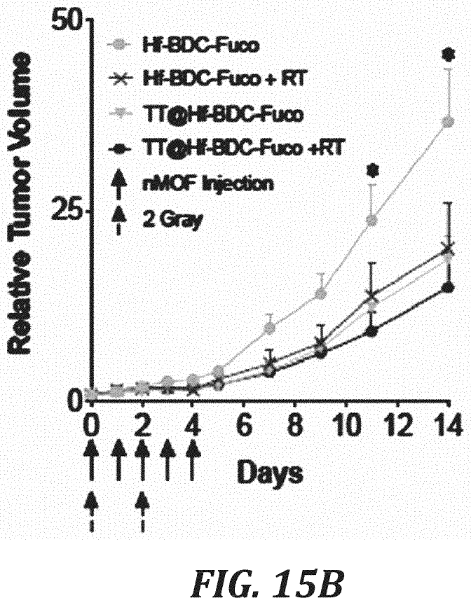

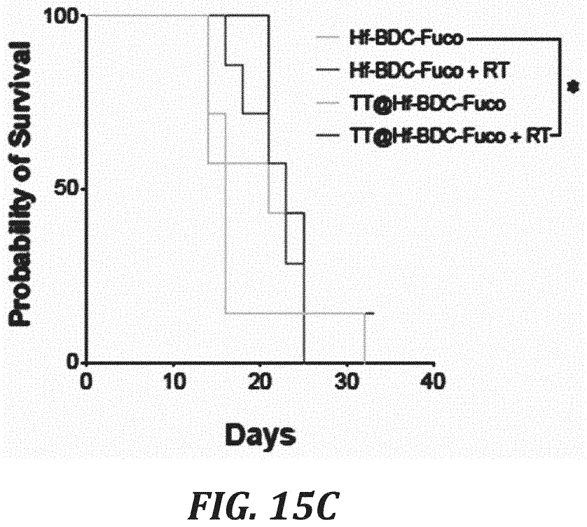

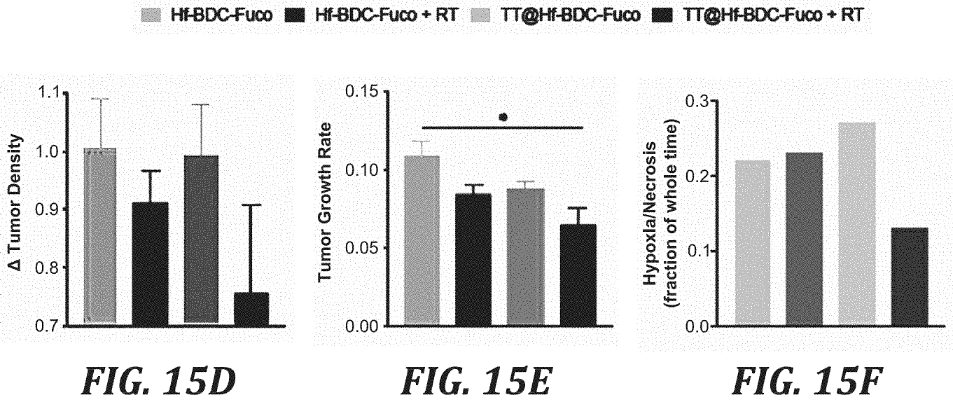

To evaluate therapeutic efficacy, tumor-bearing mice were administered Hf-BDC-Fuco or TT@Hf-BDC-Fuco twice daily, for 5 consecutive days (FIG. 15A). The first nMOF administration coincided with the first of two RT fractions of 2 Gy. Comparing tumor-growth curves, the combination of TT@Hf-BDC-Fuco+IR leads to a significant decrease in relative tumor volume and increase in survival (23 vs 16 days) compared to Hf-BDC-Fuco (FIGS. 15B and 15C). Furthermore, TT@Hf-BDC-Fuco+IR resulted in decreased tumor density, growth rate, and hypoxia/necrosis compared to the Hf-BDC-Fuco control (FIGS. 15D-15F). The only instance of complete response was observed in the TT@Hf-BDC-Fuco+IR treatment group.

In Vivo Safety

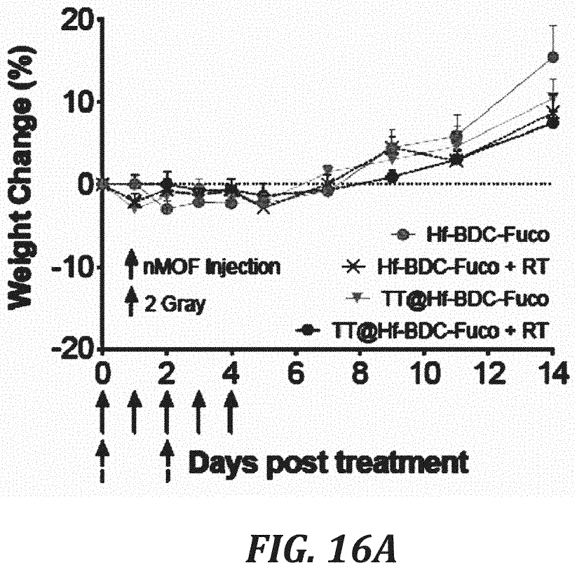

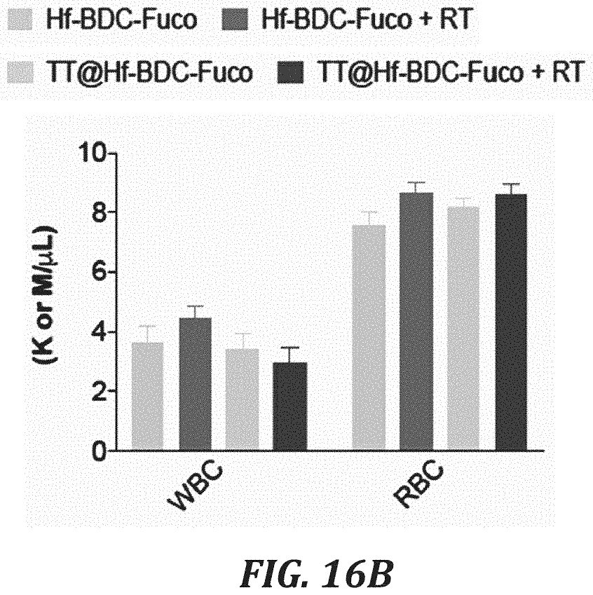

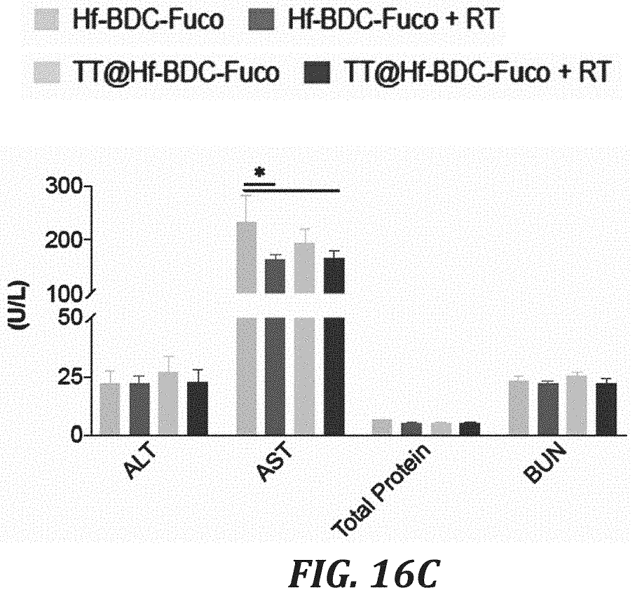

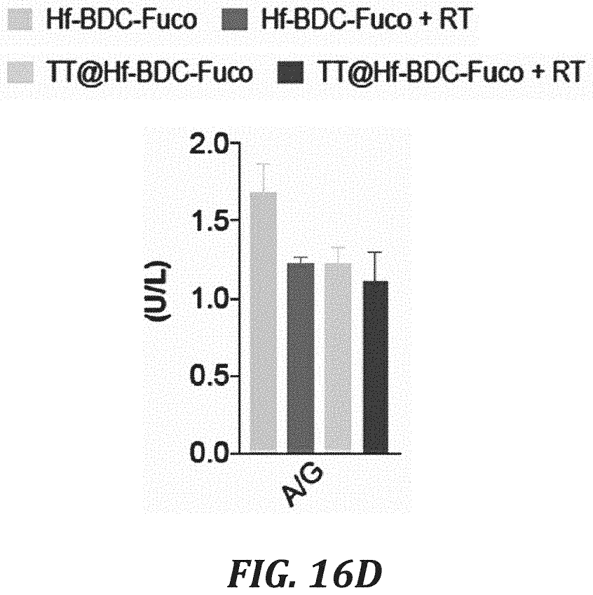

Safety evaluation of the therapeutic efficacy study did not uncover unusual changes in body or organ weight indicating good overall biocompatibility (FIG. 16A). Furthermore, complete blood count (CBC) and clinical chemistry panels indicated lack of hematologic, hepatic, or renal toxicity (FIGS. 16B and 16C).

### DISCUSSION

Drug synergy was identified across a broad range of ratios and concentrations for two exemplary chemotherapeutic agents, TMZ and Tal as used in nMOF compositions, was demonstrated herein. Subsequently, several factors were considered in the selection of an exemplary ptimal delivery vehicle and ratio of TMZ to Tal (30:1). Despite the oral bioavailability of TMZ and Tal, both agents demonstrate hydrophobic characteristics and dose-limiting toxicities which can be overcome through encapsulation. Initial Hf-BDC nMOF loading trials indicated higher loading capacity of TMZ versus Tal (data not shown). This is advantageous as prior studies found synergy at various ratios of TMZ in excess of Tal. Although potent, combining strong PARP-trappers, such as Tal, with TMZ may result in low tolerability. Therefore, a strategy for dual-encapsulation and spatiotemporal drug-release may enhance therapy and reduce unwanted toxicities.

Interest in tissue specific targeting agents has grown as reports have noted the limitation of nanoparticle delivery to tumors. An ideal target would have minimal baseline expression and naturally high or externally stimulated expression in target tissue. P-sel expression plays a critical role in the recruitment of inflammatory cells and may be increased in inflammatory cancers such as the CT26.wt model. Previously, high base levels of P-sel were identified in several human cancer types. Furthermore, P-sel expression can be modulated utilizing a therapeutic external modality (RT). In the present disclosure, P-sel expression and subsequent nanoparticle targeting was evaluated at select timepoints and radiation doses. P-sel expression appeared time-dependent both in vitro and in vivo. Additionally, fucoidan coating provided enhanced uptake in P-sel expressing cells or tissue regardless of IR administration over a control. However, comparing CT26.wt and bEnd.3 cells revealed interesting differences in nMOF uptake following a single dose of 2 Gy. IR stimulated increased uptake for all nMOF coatings in bEnd.3 cells, with Hf-BDC-Fuco eliciting the maximal increase. On the other hand, IR decreased uptake of Hf-BDC and Hf-BDC-Dex in CT26.wt cells while Hf-BDC-Fuco still saw an increase, possibly indicating an alteration in endocytic processing. In theory, receptor recycling pathways including P-sel, could be upregulated after IR while other nonspecific endocytic pathways may be down-regulated. Differential alterations in endosomal activity between cancer and endothelial cells may explain the selective increase in post-IR Hf-BDC-Fuc uptake in CT26 cells compared to other coatings. Studies evaluating the influence of IR on various endocytic pathways and receptor recycling may be warranted when optimizing tumoral accumulation of Fuco-modified nMOFs.

To gain further insight into the radiobiologic influence of treatment, radiation enhancement ratio (RER) values were calculated and graphed. Here, RER>1 indicates increased efficacy and RER<1 indicates decreased efficacy. Hf-BDC-Fuco's radioenhancing ability is demonstrated at 4 and 5 Gy (FIG. 5F), stronger radioenhancement at lower IR doses may be elicited through nMOF concentration optimization. Tal@Hf-BDC-Fuco demonstrates increasing radioenhancement with increasing IR dose. The influence of Tal is clearly evident as TMZ@Hf-BDC-Fuco only demonstrates slight enhancement at the highest IR dose. This may be a result of Tal's ability to inhibit DNA damage repair and/or its ability to induce G2/M phase cell cycle arrest, as others have found that combining Tal or Tal/TMZ with radiation increases the fraction of cells stalled in G2/M phase. In addition, the increased efficacy of TT@Hf-BDC-Fuco may arise due to the chemopotentiating role of PARP-trapping when Tal and TMZ are combined. The focus of PARPi's has shifted from application in BRCA deficient models to wider utility and these results further confirm the ability of Tal to act as a radiosensitizer in a BRCA proficient model.

One of the most promising results of TT@Hf-BDC-Fuco+IR was the complete response of a mouse treated with this therapeutic regimen. In a previously reported study by Baldwin et al. 40% of mice treated with a nano-formulation of Tal (NanoTLZ) and oral TMZ saw a complete response. In comparison, the cure rate for the present study was 14% for TT@Hf-BDC-Fuco+IR. Comparing cumulative doses of Tal and TMZ between these studies demonstrates similar Tal dose (3 vs. 3.6 mg/kg) and a 5.5-fold reduction in TMZ dose (250 vs 44 mg/kg). Total dose could be increased to enhance therapeutic efficacy with this system, however, the dose and regimen were selected based on reports of high toxicity for combined TMZ/Tal regimens. Although a reduced dose was selected, it is worth noting that a similar free drug regimen (5 mg/kg TMZ 1×5 d and 0.15 mg/kg Tal 2×5 d, repeated every 14 d) did not result in any complete responses. Importantly, as compared to the pilot study, dosing was not re-initiated 7 days after the final treatment day in the efficacy study due to unforeseen circumstances (COVID-19 restrictions), which may account for the lack of an increase in survival and complete responses.

Further evidence of enhanced efficacy was provided by histological evaluation of H&E stained tumor tissues. Here, histology revealed that 14- and 21-days post-treatment initiation, the development a hypoxic/necrotic core was reduced for TT@Hf-BDC-Fuco+IR (FIG. 16F). It is well-understood that hypoxia decreases the efficacy of therapeutic interventions including RT and chemotherapy. As such, the ability of TT@Hf-BDC-Fuco+IR to decrease hypoxia warrants further investigation. Decreased hypoxia in the TT@Hf-BDC-Fuco+IR treatment may be a result of vascular normalization, which is supported by the observed increase in CD31 expression 24 hrs after a dose of 6 Gy. This correlates well with data that radiation can cause vascular normalization and increased therapeutic delivery to tumors. While RT-induced vascular normalization could increase therapeutic efficacy (increased delivery) and minimize chemoresistance (decreased hypoxia), a simultaneous increase in P-sel expression would lead to an even greater amount of therapeutic delivery and efficacy.

In addition to a 14% cure rate, a significantly reduced tumor volume and tumor growth rate, increased survival, and evidence of decreased tumor density were observed. Future studies may benefit from alterations such as reduced administration (once daily while maintaining daily dose from b.i.d. schedule), increased radiation dose (either per fraction or number of fractions), repeated dosing, and/or combinations with immunotherapeutic agents. Furthermore, as this study was performed with a heterotropic colorectal allograft, future studies exploring the therapeutic efficacy in an orthotopic colorectal model are encouraged. With proper optimization of these parameters, this therapeutic regimen may achieve a higher rate of complete responses.

While RT has long been a mainstay for the treatment of cancer, the need to improve outcomes through multimodality treatment is ever increasing. The present disclosure demonstrates the application of the HF-BDC nMOF for use as a radioenhancing and drug delivery vehicle. Fucoidan surface functionalization provided enhanced nMOF accumulation over multiple controls in vitro and in vivo, demonstrating the vast potential for enhanced tumor accumulation and subsequent therapeutic delivery. Furthermore, the combination of TMZ and Tal was synergistic and radio-potentiating as both free drugs and encapsulated agents in vitro and as co-encapsulated agents in vivo. When administered to tumor-bearing mice and activated with IR, these TT@Hf-BDC-Fuco nMOFs demonstrated tumor control (reduced volume, density, and growth rate) and increased survival. Finally, drug-loading and nMOF coating was facile, indicating the potential ease of scale-up for clinical application. Ultimately, this report demonstrates the value of combining Tal and TMZ with P-sel targeted, and radiation dose enhancing nMOFs for enhanced chemoradiotherapy in a heterotopic, syngeneic CRC model.

The targeted compositions disclosed herein overcome challenges associated with the passive nMOF accumulation in cancer cells. In some embodiments, the use of fucoidan as a coating for achieving enhanced nMOF accumulation.

While illustrative embodiments have been illustrated and described, it will be appreciated that various changes can be made therein without departing from the spirit and scope of the invention.

1. A composition comprising a hybrid material comprising nanosized metal-organic-framework (nMOF) and two or more therapeutic agents selected from the group consisting of DNA-targeting chemotherapeutic agents, DNA Damage Repair (DDR) inhibitors, and a combination thereof.

2. The composition of claim 1, wherein the two or more therapeutic agents comprise a combination of one or more DNA-targeting chemotherapeutic agents and one or more DNA Damage Repair (DDR) inhibitors.

3. The composition of claim 1, wherein the nanosized metal-organic-framework (nMOF) comprises Hf and 1,4-benzenedicarboxylic acid (Hf-BDC).

4. The composition of claim 1, wherein the nanosized metal-organic-framework (nMOF) is PEGylated.

5. The composition of claim 1, wherein the nanosized metal-organic-framework (nMOF) is coated with an agent that has affinity to a cell surface protein unregulated under inflammatory conditions.

6. The composition of claim 1, wherein the nanosized metal-organic-framework (nMOF) is coated with a polysaccharide.

7. The composition of claim 6, wherein the polysaccharide has affinity for P-Selectin.

8. The composition of claim 6, wherein the polysaccharide is fucoidan or P-selectin glycoprotein ligand 1.

9. The composition of claim 1, wherein the one or more DNA-targeting chemotherapeutic agents is hydrophobic.

10. The composition of claim 1, wherein the one or more one or more DNA Damage Repair (DDR) inhibitors is hydrophobic.

11. The composition of claim 1, wherein the one or more DNA-targeting chemotherapeutic agents is a platinum agent, a triazene, a nitrosourea, an alkylating hexitol, or a nitrogen mustard.

12. The composition of claim 1, wherein the one or more DNA-targeting chemotherapeutic agents is dacarbazine, procarbazine, or temozolomide.

13. The composition of claim 1, wherein the one or more one or more DNA Damage Repair (DDR) inhibitors is a tyrosine kinase inhibitor or a poly (ADP-ribose) polymerase (PARP) inhibitor.

14. The composition of claim 1, wherein the one or more one or more DNA Damage Repair (DDR) inhibitors is rucaparib, talazoparib, niraparib, or olaparib.

15. A method of treatment of cancer in a subject, comprising administering to a subject in need thereof an effective therapeutic dose of a composition of claim 1.

16. The method of claim 15, wherein the composition is administered intravenously or intratumorally.

17. The method of claim 15, wherein the method further comprises administering a therapeutically effective dose of radiation to the patient.

18. The method of claim 17, wherein the administering a therapeutically effective dose of radiation to the patient is done prior to, concurrently with, or after the administration of the composition.

19. The method of claim 15, wherein the cancer is colorectal cancer, breast cancer, head and neck cancer, esophageal cancer, gastric cancer, lung cancer, pancreatic cancer, gastric cancer, bladder cancer, cervical cancer, or a brain tumor.

20. A method of treatment of tumor in a subject, comprising administering a therapeutically effective dose of radiation to the tumor, wherein the therapeutically effective dose of radiation is a dose that results in increased expression of a cell surface protein upregulated under inflammatory conditions, and administering a therapeutically effective dose of a composition of claim 5 to the subject.
